SEQUENCE LISTING (58 sequences)

SEQ ID NO: 1 - protein, 30 aa, Homo sapien
RGSPAINVAVHVFRKAADDTWEPFASGKTS

SEQ ID NO: 2 - protein, 50 aa, Homo sapien
GPTGTGESKCPLMVKVLDAVRGSPAINVAVHVFRKAADDTWEPFASGKTS

SEQ ID NO: 3 - protein, 42 aa, Homo sapien
ESGELHGLTTEEEFVEGIYKVKALGISPFHEHAEVVFTANDS

SEQ ID NO: 4 - protein, 50 aa, Homo sapien
ESGELHGLTTEEEFVEGIYKVEIDTKSYWKALGISPFHEHAEVVFTANDS

SEQ ID NO: 5 - protein, 27 aa, Homo sapien
GPRRYTIAALLSPYSYSTTAVVTNPKE

SEQ ID NO: 6 - protein, 28 aa, Homo sapien
GPRRYTIAALLSPYSYSTTAVVTNPKEL

SEQ ID NO: 7 - protein, 16 aa, Homo sapien
RSFFSFLGEAFDGARD

SEQ ID NO: 8 - protein, 50 aa, Homo sapien
MKLLTGLVFCSLVLGVSSRSFFSFLGEAFDGARDMWRAYSDMREANYIGS

SEQ ID NO: 9 - protein, 17 aa, Homo sapien
RGPGGVWAAEAISDARE

SEQ ID NO: 10 - protein, 71 aa, Homo sapien
DKYFHARGNYDAAKRGPGGVWAAEAISDARENIQRFFGHGAEDSLADQAANWGRSGKDPNHFRPAGLPEKY

SEQ ID NO: 11 - protein, 336 aa, Homo sapien
MAWTLLLLVLLSHCTGSLSQPVLTQPSSHSASSGASVRLTCMLSSGFSVGRFSGSNSGNTATLTIDFWIRWYQQKPGNPPRYLLYYHSDSNKGQGSGVPSRFSGSNDASANAGILSRVKAAPSVTLFPRISGLQLEVEADYYCGTWHSNSKNVRVFGGGTKLTVLGQPKAAPSVTLFPPSSEELQANKATLVCLISDFYPGAVTVAWKAPSSEELQANKATLVCLISDFYPGAVTVAWKADSSPVKAGVETTTPSKQSNKYAASSYLSLTPEQWKSRSYSCQVTHEGSTVEKTNKYAASSYLSLTPEQWKSHKSYSCQVTHEGSTVEKTVAPTECS

SEQ ID NO: 12 - protein, 249 aa, Homo sapien
DIQMTQSPSSLSASVGDRVDIQMTQSPSSLSASVGDRVTITCQASQDINNYLNWYQQKPGKTPKLLIYGASNLETGVPSRFSGSGSGTDFIFTISSLQPEDIATYYCQQYDNLPLTFGGGTKVEIKRTVAAPSVFIFPPSDEQLKSGTASVVCLLNNFYPREAKVQWKVKDSTYSLSSTLTLSKADNALQSGNSQESVTEQDSKDSTYSLSNTLTLSKADYEKHKVYACEVTHQGLSSPVTKSFNRGEC

SEQ ID NO: 13 - protein, 14 aa, Homo sapien
GSPAINVAVHVFRK

SEQ ID NO: 14 - protein, 13 aa, Homo sapien
AADDTWEPFASGK

SEQ ID NO: 15 - protein, 32 aa, Homo sapien
TSESGELHGLTTEEEFVEGIYKVEIDTKSYWK

SEQ ID NO: 16 - protein, 23 aa, Homo sapien
ALGISPFHEHAEVVFTANDSGPR

SEQ ID NO: 17 - protein, 22 aa, Homo sapien
YTIAALLSPYSYSTTAVVTNPK

SEQ ID NO: 18 - protein, 14 aa, Homo sapien
SFFSFLGEAFDGAR

SEQ ID NO: 19 - protein, 9 aa, Homo sapien
EANYIGSDK

SEQ ID NO: 20 - protein, 15 aa, Homo sapien
GPGGVWAAEAISDAR

SEQ ID NO: 21 - protein, 21 aa, Homo sapien
FFGHGAEDSLADQAANEWGRS

SEQ ID NO: 22 - protein, 17 aa, Homo sapien
LTCMLSSGFSVGDFWIR

SEQ ID NO: 23 - protein, 11 aa, Homo sapien
WYQQKPGNPPR

SEQ ID NO: 24 - protein, 11 aa, Homo sapien
YLLYYHSDSNK

SEQ ID NO: 25 - protein, 9 aa, Homo sapien
GQGSGVPSR

SEQ ID NO: 26 - protein, 15 aa, Homo sapien
FSGSNDASANAGILR

SEQ ID NO: 27 - protein, 8 aa, Homo sapien
LTVLSQPK

SEQ ID NO: 28 - protein, 19 aa, Homo sapien
AAPSVTLFPPSSEELQANK

SEQ ID NO: 29 - protein, 20 aa, Homo sapien
ATLVCLISDFYPGAVTVAWK

SEQ ID NO: 30 - protein, 10 aa, Homo sapien
AGVETTTPSK

SEQ ID NO: 31 - protein, 15 aa, Homo sapien
YAASSYLSLTPEQWK

SEQ ID NO: 32 - protein, 15 aa, Homo sapien
SYSCQVTHEGSTVEK

SEQ ID NO: 33 - protein, 19 aa, Homo sapien
CDIQMTQSPSSLSASVGDR

SEQ ID NO: 34 - protein, 16 aa, Homo sapien
LLIYGASNLETGVPSR

SEQ ID NO: 35 - protein, 16 aa, Homo sapien
FSGSGSGTDFIFTISR

SEQ ID NO: 36 - protein, 12 aa, Homo sapien
TFGGGTKVEIKR

SEQ ID NO: 37 - protein, 18 aa, Homo sapien
TVAAPSVFIFPPSDEQLK

SEQ ID NO: 38 - protein, 16 aa, Homo sapien
SGTASVVCLLNNFYPR

SEQ ID NO: 39 - protein, 20 aa, Homo sapien
VDNALQSGNSQESVTEQDSK

SEQ ID NO: 40 - protein, 14 aa, Homo sapien
DSTYSLSSTLTLSK

SEQ ID NO: 41 - protein, 17 aa, Homo sapien
VYACEVTHQGLSSPVTK

SEQ ID NO: 42 - protein, 6 aa, Homo sapien
VFVFPR

SEQ ID NO: 43 - protein, 15 aa, Homo sapien
ESVTDHVNLITPLEK

SEQ ID NO: 44 - protein, 10 aa, Homo sapien
PLQNFTLCFR

SEQ ID NO: 45 - protein, 7 aa, Homo sapien
AYSDLSR

SEQ ID NO: 46 - protein, 12 aa, Homo sapien
AYSLFSYNTQGR

SEQ ID NO: 47 - protein, 8 aa, Homo sapien
DNELLVYK

SEQ ID NO: 48 - protein, 10 aa, Homo sapien
VGEYSLYIGR

SEQ ID NO: 49 - protein, 10 aa, Homo sapien
QGYFVEAQPK

SEQ ID NO: 50 - protein, 13 aa, Homo sapien
IVLGQEQDSYGGK

SEQ ID NO: 51 - protein, 11 aa, Homo sapien
GYVIIKPLVWV

SEQ ID NO: 52 - protein, 8 aa, Homo sapien
NAINNGVR

SEQ ID NO: 53 - protein, 9 aa, Homo sapien
LGTLLPLQK

SEQ ID NO: 54 - protein, 8 aa, Homo sapien
MFYIKPIK

SEQ ID NO: 55 - protein, 10 aa, Homo sapien
HGCGQYSAQR

SEQ ID NO: 56 - protein, 15 aa, Homo sapien
VHIENCDSSDPTAYL

SEQ ID NO: 57 - protein, 15 aa, Homo sapien
GSPAINVAVHVFRKK

SEQ ID NO: 58 - protein, 15 aa, Homo sapien
ASQSVSSYLAWYQQK

(19) United States
(12) Patent Application Publication
(10) Pub. No.: US 2010/0323381 A1
(43) Pub. Date: Dec. 23, 2010

(54) CLASSIFYING AMYLOIDOSIS

(76) Inventors: Harold R. Bergen, III, Spring Valley, MN (US); Steven R. Zeldenrust, Rochester, MN (US); David R. Barnidge, Rochester, MN (US); Ahmet Dogan, Rochester, MN (US); Julie A. Vrana, Rochester, MN (US); Jeffrey D. Gamez, Rochester, MN (US); Jason D. Theis, Kasson, MN (US)

Correspondence Address: FISH & RICHARDSON P.C. (TC), PO BOX 1022, MINNEAPOLIS, MN, 55440-1022, US

(21) Appl. No.: 12/866,709

(22) PCT Filed: Feb. 6, 2009
(86) PCT No.: PCT/US09/33477

§ 371 Date: Aug. 17, 2010

Related U.S. Application Data
(60) Provisional application No. 61/027,273, filed on Feb. 8, 2008.

(51) Int. Cl. C12Q 1/37

(52) U.S. Cl. 435/23

(57) ABSTRACT

This document provides methods and materials related to determining the identity of amyloid polypeptides in biological samples. For examples, methods and materials for identifying an amyloid polypeptide present in a fat aspirate are provided herein.

### BACKGROUND

1. Technical Field

This document relates to methods and materials involved in the rapid determination of amyloid polypeptides involved in amyloidosis.

2. Background Information

Human amyloidosis describes a condition where one or more of about 23 polypeptides form non-soluble pathological fibrils in various organs and tissues throughout the body including abdominal fat. These amyloid deposits give distinctive staining with Congo Red on paraffin embedded tissue sections of biopsies or subcutaneous abdominal fat aspirates. This method is combined with clinical, immunohistochemical, and genetic information to diagnose the condition and in some cases, identify the amyloid polypeptide involved.

### SUMMARY

This document provides methods and materials related to rapid determination of amyloid polypeptides in biological samples. Determining the identity of an amyloid polypeptide that is involved in amyloidosis can be achieved in a quick and reliable manner using the methods and materials provided herein. For example, protease digested (e.g., trypsin digested) fat aspirates can be assessed using mass spectrometry (e.g., nanoRPLC-ESI-tandem mass spectrometry) to determine the identity of one or more amyloid polypeptides, without performing HPLC or Western blotting. In some cases, laser microdissection (LMD) can be combined with nanoRPLC-ESI-tandem mass spectrometry to identify one or more amyloid polypeptides in a biological sample. In some cases, the methods and materials provided herein can be used to prepare and perform trypsin digestion on samples from formalin-fixed, paraffin-embedded (FFPE) tissue sections or fat aspirates to establish robust identification of amyloid polypeptides.

In general, one aspect of this document features a method for evaluating amyloidosis in a mammal. The method comprises determining the identity of an amyloid polypeptide present in a sample (e.g., a fat aspirate sample) from the mammal using mass spectrometry, wherein the sample (e.g., fat aspirate sample) was processed using reverse phase liquid chromatography before performing the mass spectrometry. In some cases, the method can comprise determining the identity of an amyloid polypeptide present in a sample (e.g., a fat aspirate sample) from the mammal using mass spectrometry, wherein the sample (e.g., fat aspirate sample) was not processed using liquid chromatography before performing the mass spectrometry. The mammal can be a human. The liquid chromatography can be high-performance liquid chromatography. The amyloid polypeptide can be a transthyretin (TTR) polypeptide, a serum amyloid-associated (SAA) polypeptide, an immunoglobulin light chain lambda (IGL) polypeptide, an immunoglobulin light chain kappa (IGK) polypeptide, a serum amyloid P (SAP) polypeptide, a LECT2 polypeptide, an immunoglobulin gamma (-1-4) chain C polypeptide, an immunoglobulin alpha (-1-2) chain C polypeptide, an immunoglobulin delta chain C polypeptide, an immunoglobulin mu heavy chain polypeptide, an immunoglobulin heavy chain polypeptide, a serum amyloid A-4 protein precursor polypeptide, a fibrinogen alpha polypeptide, gelsolin, a beta2 microglobulin polypeptide, an apoplipoprotein AI polypeptide or AII polypeptide, or a lysozyme polypeptide. The fat aspirate sample can be a sample treated with a protease before the mass spectrometry. The protease can be trypsin.

In another aspect, this document provides a method for evaluating amyloidosis in a mammal. The method comprises (a) determining whether or not a tissue sample Congo Red positive for amyloid from the mammal comprises a transthyretin polypeptide using mass spectrometry, (b) determining whether or not a tissue sample Congo Red positive for amyloid from the mammal comprises a serum amyloid A polypeptide using mass spectrometry, (c) determining whether or not a tissue sample Congo Red positive for amyloid from the mammal comprises a lambda light chain polypeptide using mass spectrometry, and (d) determining whether or not a tissue sample Congo Red positive for amyloid from the mammal comprises a kappa light chain polypeptide using mass spectrometry. The method can comprise determining whether or not a tissue sample Congo Red positive for amyloid from the mammal comprises a LECT2 polypeptide using mass spectrometry. The mammal can be a human. The tissue sample can be treated with a protease before the mass spectrometry. The protease can be trypsin.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 contains a list of results from heart biopsy tissue.

FIG. 2 contains a list of results from heart biopsy tissue.

FIG. 3 contains a list of results from kidney biopsy tissue.

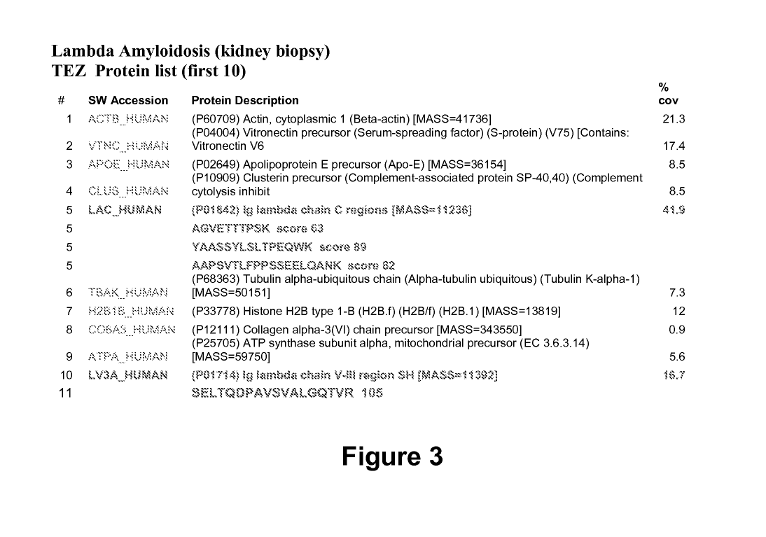

FIG. 4 contains a list of results from kidney biopsy tissue.

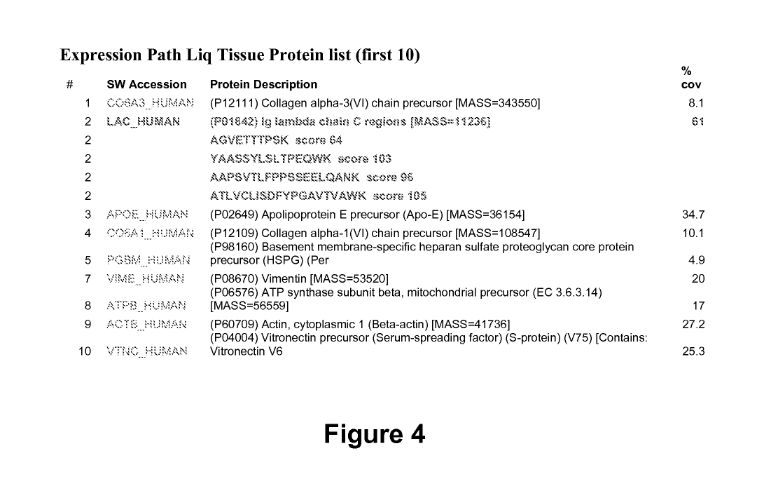

FIG. 5 contains a list of results from fat aspirate.

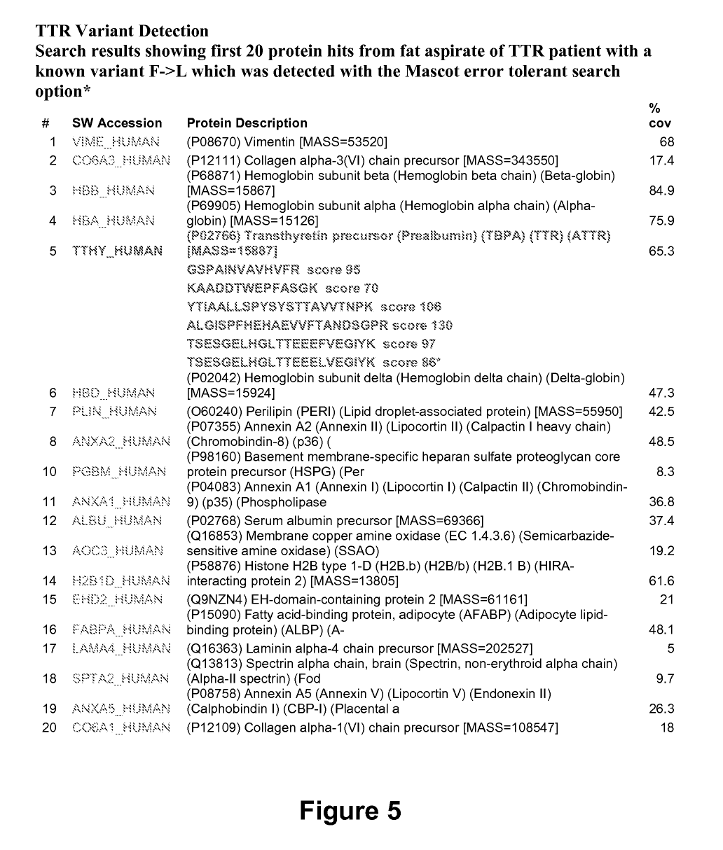

FIG. 6 contains a list of results additional amyloid identifications.

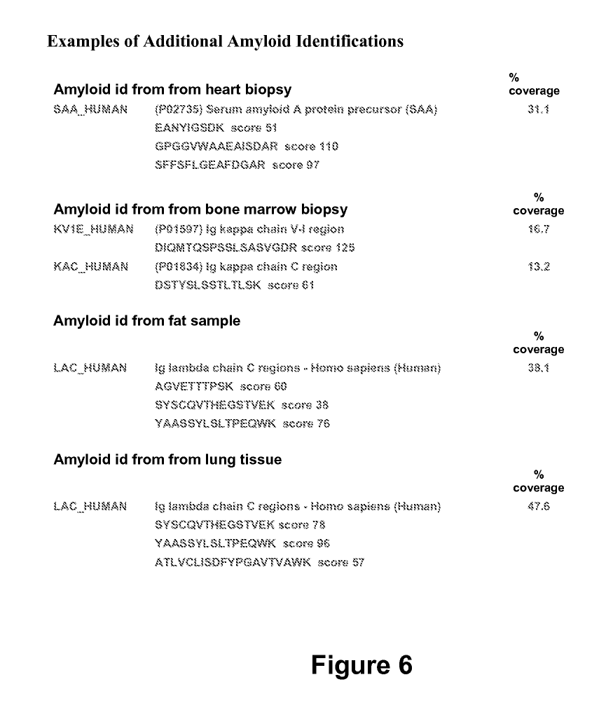

FIG. 7. Amyloid areas are identified through Congo Red positivity observed by transmitted light (left image) or fluorescence (right image) (210× magnification).

FIG. 8 is a photograph of a microdissection using a Leica Microdissection System (LMD6000). The amyloid area to be captured is outlined. 210× magnification.

FIGS. 9A and 9B are photographs of the laser cuts along the line drawn. The tissue drops into a microfuge cap containing buffer (either Tris/EDTA/0.002% Zwittergent 3-16 or Expression Pathology Liquid Tissue).

FIG. 10 is a photograph confirming that tissue was collected. The microfuge tube cap was observed under 42× magnification. The tissue in solution was heated for 90 minutes at 98° C. with vortexing/centrifuging every 20 minutes. Samples were sonicated for 30 minutes and then digested with 1 μg of trypsin for 16-18 hours at 37° C. After reduction by DTT, the sample was analyzed using a Thermo LTQ-Orbitrap mass spectrometer. Polypeptide sequences were identified using Mascot software.

FIG. 11 is a photograph of a heart biopsy in which immunohistochemistry indicated the tissue was TTR positive (210× magnification). Mass spectrometry identified the following TTR tryptic polypeptides in this specimen. Six different SAP peptides were also identified. No other amyloid polypeptides were observed. The underlined portions of the MS peptides matched the TTR sequence:

(SEQ ID NO: 1)
1. RGSPAINVAVHVFRKAADDTWEPFASGKTS
(SEQ ID NO: 2)
2. GPTGTGESKCPLMVKVLDAVRGSPAINVAVHVFRKAADDT-
WEPFASGKTS
(SEQ ID NO: 3)
3. ESGELHGLTTEEEFVEGIYKVKALGISPFHEHAEVVFTANDS
(SEQ ID NO: 4)
4. ESGELHGLTTEEEFVEGIYKVEIDTKSYWKALGISPFHEH-
AEVVFTANDS
(SEQ ID NO: 5)
5. GPRRYTIAALLSPYSYSTTAVVTNPKE
(SEQ ID NO: 6)
6. GPRRYTIAALLSPYSYSTTAVVTNPKEL.

FIG. 12 is a photograph of a heart biopsy in which immunohistochemistry indicated the tissue was SAA positive (210 × magnification). Mass spectrometry identified the following SAA tryptic polypeptides in this specimen. No other amyloid polypeptides were observed. The underlined portions of the MS peptides matched the SAA sequence:

(SEQ ID NO: 7)
1. RSFFSFLGEAFDGARD
(SEQ ID NO: 8)
2. MKLLTGLVFCSLVLGVSSRSFFSFLGEAFDGARDMWRA-
YSDMREANYIGS
(SEQ ID NO: 9)
3. RGPGGVWAAEAISDARE
(SEQ ID NO: 10)
4. DKYFHARGNYDAAKRGPGGVWAAEAISDARENIQRFFGH-
GAEDSLADQAANWGRSGKDPNHFRPAGLPEKY

FIG. 13 is a photograph of a liver biopsy in which immunohistochemistry indicated tissue was Lambda light chain positive (210× magnification). Mass spectrometry identified the following lambda light chain tryptic polypeptides in this specimen. Three different SAP peptides were also identified. No other amyloid polypeptides were observed. The underlined portions of the MS peptides matched the Locus S25755 Human Ig Lambda chain sequence:

(SEQ ID NO: 11)
1. MAWTLLLLVLLSHCTGSLSQPVLTQPSSHSASSGASVRLT-
CMLSSGFSVGRFSGSNSGNTATLTIDFWIRWYQQKPGNPPRYLLYYHSDS
NKGQGSGVPSRFSGSNDASANAGILSRVKAAPSVTLFPRISGLQLEVEAD
YYCGTWHSNSKNVRVFGGGTKLTVLGQPKAAPSVTLFPPSSEELQANKAT
LVCLISDFYPGAVTVAWKAPSSEELQANKATLVCLISDFYPGAVTVAWK
ADSSPVKAGVETTTPSKQSNKYAASSYLSLTPEQWKSRSYSCQVTHEGST
VEKTNKYAASSYLSLTPEQWKSHKSYSCQVTHEGSTVEKTVAPTECS

FIG. 14 is a photograph of a bone marrow biopsy in which immunohistochemistry indicated the tissue was Kappa light chain positive (210× magnification). Mass spectrometry identified the following kappa light chain tryptic polypeptides in this specimen. Two different SAP polypeptides were also identified. No other amyloid polypeptides were observed. The underlined portions of the MS peptides matched the Locus AAD29608 Human Kappa 1 Ig light chain sequence:

(SEQ ID NO: 12)
1. DIQMTQSPSSLSASVGDRVDIQMTQSPSSLSASVGDRVTITCQAS-
QDINNYLNWYQQKPGKTPKLLIYGASNLETGVPSRFSGSGSGTDFIFTI
SSLQPEDIATYYCQQYDNLPLTFGGGTKVEIKRTVAAPSVFIFPPSDEQ
LKSGTASVVCLLNNFYPREAKVQWKVKDSTYSLSSTLTLSKADNALQSG
NSQESVTEQDSKDSTYSLSNTLTLSKADYEKHKVYACEVTHQGLSSPVT
KSFNRGEC.

FIG. 15. Extracellular proteinaceous deposition with crystals (Case 6). Aggregates of eosinophilic proteinaceous deposits with crystal formation are surrounded by a dense neoplastic lymphoplasmacytic infiltrate (A). Immunohistochemical stains demonstrate kappa light chain (B) immunostaining but absent lambda (C) consistent with kappa light chain restriction. The latter was also demonstrated by mass spectrometry, which in addition revealed Ig gamma and Ig heavy chain peptides.

FIG. 16. Extracellular proteinaceous deposition in the brain in a case of splenic marginal zone lymphoma. Lakes of eosinophilic deposits lacking crystals are involving predominantly white matter (A), although scant smaller deposits are also present in gray matter (B). Immunohistochemical stains demonstrate kappa light chain expression (C) but not lambda (D), as well as IgM.

FIG. 17. Amyloidoma of sciatic nerve and laser microdissection (case 12). Localized tumefactive amyloid deposit replacing a peripheral nerve fascicle demonstrated on H&E (A) and Congo Red (B) stains. Rhodamine optics demonstrates bright red fluorescence (C, Congo Red). Several areas are traced in the computer screen, microdissected and submitted for analysis using available software. Lambda light chain restriction was demonstrated by immunohistochemistry and mass spectrometry.

FIG. 18. Crystal storing histiocytosis (case 7). Intracellular refractile crystals are evident on H&E stain (A). The histiocytic nature of the cells is confirmed by strong CD68 expression (B). In situ hybridization demonstrates kappa mRNA (but not lambda) focally in perivascular plasma cells (C and D, respectively). Although immunohistochemical stains for kappa and lambda immunoglobulin light chains were non-contributory because of a high level of background staining, mass spectrometry demonstrated the presence of kappa light chain only, consistent with kappa light chain restriction.

FIG. 19. Tandem mass spectrometry (MS) analysis identifies kappa light chain V-III in case 9. The Orbitrap MS survey scan containing a parent ion mass of 916.943 Da is shown in panel A. The LTQ MS/MS scan of the doubly charged ion at 916 Da is shown in panel B. A data base search against the MS/MS spectra revealed this to be ASQSVSSYLAWYQQK (SEQ ID NO:58) with an XCorr of 5.14 and −1.9 ppm mass error corresponding to Ig kappa light chain (KV312_HUMAN).

### DETAILED DESCRIPTION

This document provides methods and materials related to identifying amyloid polypeptides in biological samples (e.g., tissue biopsy or fat aspirates). Determining the identity of an amyloid polypeptide that is involved in amyloidosis can be achieved in a quick and reliable manner using the methods and materials provided herein.

A biological sample can be obtained from any mammal having amyloidosis or suspected of having amyloidosis. Examples of such mammals include, humans, non-human primates, dogs, cats, rats, or mice. A biological sample can be a tissue biopsy (e.g., a laser dissected tissue biopsy) or a fat aspirate. For example, a biological sample can be obtained from a subcutaneous fat aspirate and can contain pieces of adipose tissue. In some cases, abdominal fat aspirate can be taken with a 18 gauge needle and syringe, and can be compressed between two slides that can then be pried apart and briefly formalin fixed and Congo Red stained. The presence of amyloid can be identified by positive Congo Red staining

Once an amyloid positive specimen is obtained, the sample can be processed as described herein. For example, a tissue biopsy or fat aspirate can be treated with a protease (e.g., trypsin) to create polypeptide fragments. The digested sample can be analyzed using mass spectrometry to identify any amyloid polypeptides present. Examples of amyloid tryptic polypeptides and deviations generated via protease treatment (e.g., trypsin treatment) include, without limitation, TTR polypeptides (e.g., GSPAINVAVHVFRK (SEQ ID NO:13); AADDTWEPFASGK (SEQ ID NO:14); TSESGELHGLTTEEEFVEGIYKVEIDTKSYWK (SEQ ID NO:15); ALGISPFHEHAEVVFTANDSGPR (SEQ ID NO:16); and YTIAALLSPYSYSTTAVVTNPK (SEQ ID NO:17)), SAA polypeptides (e.g., SFFSFLGEAFDGAR (SEQ ID NO:18); EANYIGSDK (SEQ ID NO:19); GPGGVWAAEAISDAR (SEQ ID NO:20); and FFGHGAEDSLADQAANEWGRS (SEQ ID NO:21)), IGL polypeptides (e.g., LTCMLSSGFSVGDFWIR (SEQ ID NO:22); WYQQKPGNPPR (SEQ ID NO:23); YLLYYHSDSNK (SEQ ID NO:24); GQGSGVPSR (SEQ ID NO:25); FSGSNDASANAGILR (SEQ ID NO:26); LTVLSQPK (SEQ ID NO:27); AAPSVTLFPPSSEELQANK (SEQ ID NO:28); ATLVCLISDFYPGAVTVAWK (SEQ ID NO:29); AGVETTTPSK (SEQ ID NO:30); YAASSYLSLTPEQWK (SEQ ID NO:31); and SYSCQVTHEGSTVEK (SEQ ID NO:32)), IGK polypeptides (e.g., CDIQMTQSPSSLSASVGDR (SEQ ID NO:33); LLIYGASNLETGVPSR (SEQ ID NO:34); FSGSGSGTDFIFTISR (SEQ ID NO:35); TFGGGTKVEIKR (SEQ ID NO:36); TVAAPSVFIFPPSDEQLK (SEQ ID NO:37); SGTASVVCLLNNFYPR (SEQ ID NO:38); VDNALQSGNSQESVTEQDSK (SEQ ID NO:39); DSTYSLSSTLTLSK (SEQ ID NO:40); and VYACEVTHQGLSSPVTK (SEQ ID NO:41)), SAP polypeptides (e.g., VFVFPR (SEQ ID NO:42); ESVTDHVNLITPLEK (SEQ ID NO:43); PLQNFTLCFR (SEQ ID NO:44); AYSDLSR (SEQ ID NO:45); AYSLFSYNTQGR (SEQ ID NO:46); DNELLVYK (SEQ ID NO:47); VGEYSLYIGR (SEQ ID NO:48); QGYFVEAQPK (SEQ ID NO:49); IVLGQEQDSYGGK (SEQ ID NO:50); and GYVIIKPLVWV (SEQ ID NO:51)), and LECT2 polypeptides (e.g., NAINNGVR (SEQ ID NO:52); LGTLLPLQK (SEQ ID NO:53), MFYIKPIK(SEQ ID NO:54), HGCGQYSAQR (SEQ ID NO:55), and VHIENCDSSDPTAYL (SEQ ID NO:56). Protease-cleaved polypeptides (e.g., tryptic polypeptides) from other amyloid subtypes such as immunoglobulin gamma (-1-4) chain C, immunoglobulin alpha (-1-2) chain C, immunoglobulin delta chain C, immunoglobulin mu heavy chain, immunoglobulin heavy chain, serum amyloid A-4 protein precursor, fibrinogen alpha, apolipoprotein A (I and II), and gelsolin can also be identified.

LC-MS/MS analysis can be performed by any appropriate method. One method for polypeptide identification can include using nano-flow liquid chromatography electrospray tandem mass spectrometry (nanoLCESI-MS/MS) using a ThermoFinnigan LTQ Orbitrap Hybrid Mass Spectrometer (ThermoElectron Bremen, Germany) coupled to an Eksigent nanoLC-2D HPLC system. Other mass spectrometry methods can be used for sample analysis such as the low flow liquid chromotography ABI 5000 Triple Quadrupole Mass Spectrometer (lowLCESI-MS/MS). This method uses labeled polypetides as internal controls as described below. The resulting data can be interpreted using appropriate software (e.g., Analyst Software from ABI).

In some cases, a polypeptide can be synthesized and used as an internal mass spectrometry control to, for example, aid in the identification of a particular protease-cleaved polypeptide present within a sample. For example, one or more (e.g., two, three, four, five, six, seven, or more) different polypeptides can be synthesized and used as an internal control. Such a polypeptide can have the sequence set forth in any one of SEQ ID NOs:1-56. In some cases, a polypeptide for use as an internal control can be labeled. For example, a polypeptide having the sequence set forth in SEQ ID NO:13 can include two isotopically labeled lysine residues such that the polypeptide is GSPAINVAVHVFRKK (SEQ ID NO:57), where the two underlined lysine residues are labeled with ¹³C, ¹⁵N, or both.

This document also provides methods and materials to assist medical or research professionals in determining whether or not a mammal has a particular form of amyloidosis. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the presence or absence of one or more amyloid polypeptides in a biological sample obtained from a mammal, and (2) communicating information about an amyloidosis condition of the mammal to that professional. Such information can include a diagnosis of a particular form of amyloidosis. Examples of different forms of amyloidosis include, without limitation, AL, ATTR, AA, AH, Agel, Afib, AApoA-I, and AApoA-II.

After the presence or absence of one or more amyloid polypeptides is reported, a medical professional can take one or more actions that can affect patient care. For example, a medical professional can record the presence or absence of one or more amyloid polypeptides for a patient's biological sample in a patient's medical record. In some cases, a medical professional can record a diagnosis of amyloidosis, or otherwise transform the patient's medical record, to reflect the patient's medical condition. In some cases, a medical professional can review and evaluate a patient's entire medical record, and assess multiple treatment strategies, for clinical intervention of a patient's condition.

A medical professional can initiate or modify treatment for amyloidosis symptoms after receiving information regarding the amyloid polypeptides present within a patient's biological sample. In some cases, a medical professional can compare previous reports of an amyloid polypeptide content with a recently determined amyloid polypeptide content, and recommend a change in therapy. In some cases, a medical professional can enroll a patient in a clinical trial for novel therapeutic intervention of amyloidosis. In some cases, a medical professional can elect waiting to begin therapy until the patient's symptoms require clinical intervention. Examples of amyloidosis treatments include, without limitation, organ transplantation and drug therapy.

A medical professional can communicate the amyloid polypeptide content within a sample to a patient or a patient's family. In some cases, a medical professional can provide a patient and/or a patient's family with information regarding amyloidosis, including treatment options, prognosis, and referrals to specialists. In some cases, a medical professional can provide a copy of a patient's medical records to communicate the amyloid polypeptide content within a sample to a specialist.

A research professional can apply information regarding the amyloid polypeptide content within a sample from a mammal to advance amyloidosis research. For example, a researcher can compile data on the amyloid polypeptide content within a sample with information regarding the efficacy of a drug for treatment of amyloidosis symptoms to identify an effective treatment. In some cases, a research professional can determine the amyloid polypeptide content within a sample to evaluate a subject's enrollment, or continued participation in a research study or clinical trial. In some cases, a research professional can classify the severity of a subject's condition, based on the content of amyloid polypeptides within a sample.

Any appropriate method can be used to communicate information to another person (e.g., a professional). For example, information can be given directly or indirectly to a professional. For example, a laboratory technician can input the amyloid polypeptide content within a sample into a computer-based record. In some cases, information is communicated by making an physical alteration to medical or research records. For example, a medical professional can make a permanent notation or flag a medical record for communicating a diagnosis to other medical professionals reviewing the record. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

The invention will be further described in the following examples, which do not limit the scope of the invention described in the claims.

### Examples

### Example 1

### Preparing Fat Aspirate for Mass Spectrometry

The following reagents and supplies were used: gloves; Netwell plates (Corning Netwells, Cat. #3477 or Fisher Scientific, Cat. #07-200-211); RPMI1640 with Phenol Red (PR; Sigma, Cat. #R0883—500 mL); RPMI1640 without PR (Sigma, Cat. #R7509—500 mL); 1000 U/mL Heparin sodium salt stock solution (Sigma (Fluka), Cat. #H4784—250 mg); 1% Sodium Azide stock solution (Fischer Scientific, Cat. #S227-25); 0.1M TRIS ph 8.0; small ice bath; 1.5 mL screw top microcentrifuge tubes (Sarstedt, Cat. #72.692.005); Qiagen Buffer EL (Erythrocyte Lysis buffer; Cat. #79217 for 1 L); Acetone ART 100E pipet tips; Hexafluoro-2-propanol (HFP; Sigma, Cat. #105228-25G); Zwittergent 3-16 Detergent (Calbiochem; Cat. #693023, lot B62555); Trypsin (Promega, Cat. #V5111 100 μg); DTT (Sigma, Cat. #43816-10 mL; 1M solution); 0.22 μm filter Corning 500 mL filter system #430769, and Millipore Steriflip 0.22 μm filter. The following equipment was used: Eppendorf Thermomixer at 37° C. or Isotemp 205 Waterbath@37° C.; Branson 1210 waterbath sonicator; and a microcentrifuge. Fat tissue is a common site of amyloid deposition. The following procedure was used to process and digest fat aspirates for mass spectrometry analysis. Fat aspirates are taken from the anterior abdominal wall using a large bore needle. Each sample was placed into a tube containing RPMI1640 PR+ with 10 U/mL heparin and 0.03% sodium azide. The tube containing the fat aspirate sample was vortexed briefly to resuspend the fat tissue. The Netwell nylon filter was prewet with media, and the pieces of fat suspended in media were poured onto a Corning Netwell nylon filter labeled with a patient identifier. The fat tissue was rinsed with RPMI1640 PR free with 10 U/mL heparin and 0.03% NaN₃ followed by Qiagen EL buffer. The Netwell containing fat was incubated in EL buffer on ice for 15 minutes. Then, the fat tissue was rinsed with RPMI1640 solution. A piece of fat was removed from the Netwell using a 100E pipet tip and placed into a 1.5 mL microcentrifuge tube. The liquid was removed and the fat tissue allowed to dry. HFP containing 0.002% zwittergent 3-16 was added to each tube. The tubes were vortexed and sonicated and allowed to air dry. The fat was resuspended in 0.1M Tris pH 8.0 which was then vortexed and sonicated. To digest the samples, trypsin was added, and the tubes were mixed and incubated at 37° C. overnight with occasional vortexing. 0.1M DTT solution was added to each sample and heated for 5 minutes at 95-98° C. The samples were either stored at −20° C. or directly analyzed via mass spectrometry.

### Example 2

### Rapid and Sensitive Identification of Amyloid Polypeptides from Formalin-Fixed Paraffin-Embedded Sections and Fat Aspirates: Clinical Diagnosis of Systemic Amyloidosis

The study used FFPE tissues and fat aspirates from known cases of amyloidosis, previously characterized as transthyretin (TTR), serum amyloid-associated protein (SAA), and immunoglobulin light chain lambda (IGL) and Kappa (IGK). Laser microdissection was used to capture the Congo Red stained amyloid plaque enriched regions of FFPE sections or formalin fixed fat aspirates. Digested fat tissues were analyzed with a ThermoFinnigan LTQ Orbitrap Hybrid Mass Spectrometer (ThermoElectron, Bremen, Germany) coupled to an Eksigent nanoLC-2D HPLC system (Eksigent, Dublin, Calif.). The equivalent of 4 μL of the digest peptide mixture was loaded onto a 250 nL OPTI-PAK trap (Optimize Technologies, Oregon City, Oreg.) custom packed with Michrom Magic C8 solid phase (Michrom Bioresources, Auburn, Calif.) and eluted with an acetonitrile gradient (A=0.2% formic acid, B=80% acetonitrile/5% isopropanol/0.2% formic acid) through a Michrom packed tip capillary Magic C18 column (75 μm×150 mm). The LTQ-Orbitrap mass spectrometer was operated in data-dependent mode to automatically switch between LTQ (MS) and LTQ-Orbitrap (MS/MS) acquisition. Survey full scan MS spectra (from 375-1800) were acquired. The most intense ions (up to five, depending on signal intensity) were sequentially isolated for fragmentation in the linear ion trap using collisionally induced dissociation at a target value of 100,000 charges. The resulting fragment ions were recorded in the Orbitrap with a resolution 60,000 at m/z 400. The MS/MS raw data were converted to DTA files using extract_msn.exe from Bioworks 3.2 and correlated to theoretical fragmentation patterns of tryptic peptide sequences from the Swissprot databases. Proteins corresponding to these polypeptides were identified using Scaffold software (Proteome Software Inc., Portland, Oreg.) (includes Mascot, XTandem, Seaquest algorithms). All searches were conducted with variable modifications allowing carbamidomethylation of cysteine, oxidation of methionines for methione sulphoxide, and protein N-terminal acetylation. The search was restricted to both full and partial trypsin generated peptides allowing for up to three missed cleavages and was restricted to human.

Peptide mass search tolerances were set to 20 ppm, and fragment mass tolerance were set to ±1.00 Daltons. Polypeptide identifications were accepted if they could be established at greater than 90.0% probability as specified by the Peptide Prophet algorithm. Protein identifications were accepted if they could be established at greater than 90.0% probability and contained at least one identified polypeptide. Protein probabilities were assigned by the Protein Prophet algorithm.

### Slide Prep and Laser Microdissection

10 μm sections of FFPE tissue were cut onto either Leica PEN slides or Expression Pathology DIRECTOR energy transfer slides for laser microdissection. Slides were air dried and then melted in a 70° C. oven. Tissue slides were deparaffinized and rehydrated through xylene, absolute ethanol, 95% ethanol and water. Slides were stained in a progressive hematoxylin, rinsed in running water, and stained for Congo Red using a modified Puchtler's method. The amyloid areas were Congo Red positive as observed by transmitted light or fluorescence. Using a Leica Microdissection System (LMD6000), the amyloid area to be captured was outlined.

The laser cuts along a line drawn and the tissue drops into a 500 μL microfuge cap containing buffer (either Tris/EDTA/0.002% Zwittergent 3-16 or Expression Pathology Liquid Tissue®). The region where the tissue was cut was viewed by transmitted light or fluorescence. Viewing the microfuge tube cap under 42× magnification revealed the tissue sample that was collected). The tubes were capped and vortexed to transfer cap contents to bottom of tube.

### Processing and Trypsin Digestion

Initial attempts to identify the amyloid polypeptides present in tissue utilized the Expression Pathology Liquid Tissue® MS Protein Prep Kit, following their instructions. Briefly, the 20 μL Liquid Tissue® captured samples were heated for 90 minutes at 95° C. and vortexed. 1 μL of the trypsin solution was added, and the samples incubated overnight at 37° C. Then, 2 μL of 100 mM DTT was added, followed with heating at 95° C. for 5 minutes. The samples were stored at −80° C. until analysis. This procedure was modified to collect the microdissection tissues in 35 μL 10 mM Tris, pH8.0/1 mM EDTA/0.002% Zwittergent 3-16® (TEZ). The samples are heated for 90 minutes at 98° C. with intermittent vortexing, and then sonicated for 30 minutes. 1.5 μg trypsin were added to the samples that were then incubated overnight at 37° C., reduced with 3 μL 100 mM DTT at 95° C. for 5 minutes, and stored at −80° C. Other co-solvents were tested such as acetonitrile, hexafluroisopropanol (HFIP), and DMSO. All nanoRPLC-ESI-tandem mass spectrometry analyses were performed by diluting 5 μL digest sample in 20 μL of 0.15% formic acid/0.05%TFA and trap injecting 10 or 20 μL.

Examples of Mascot Search Outputs from Two Different Patient Cases, TTR and Lambda

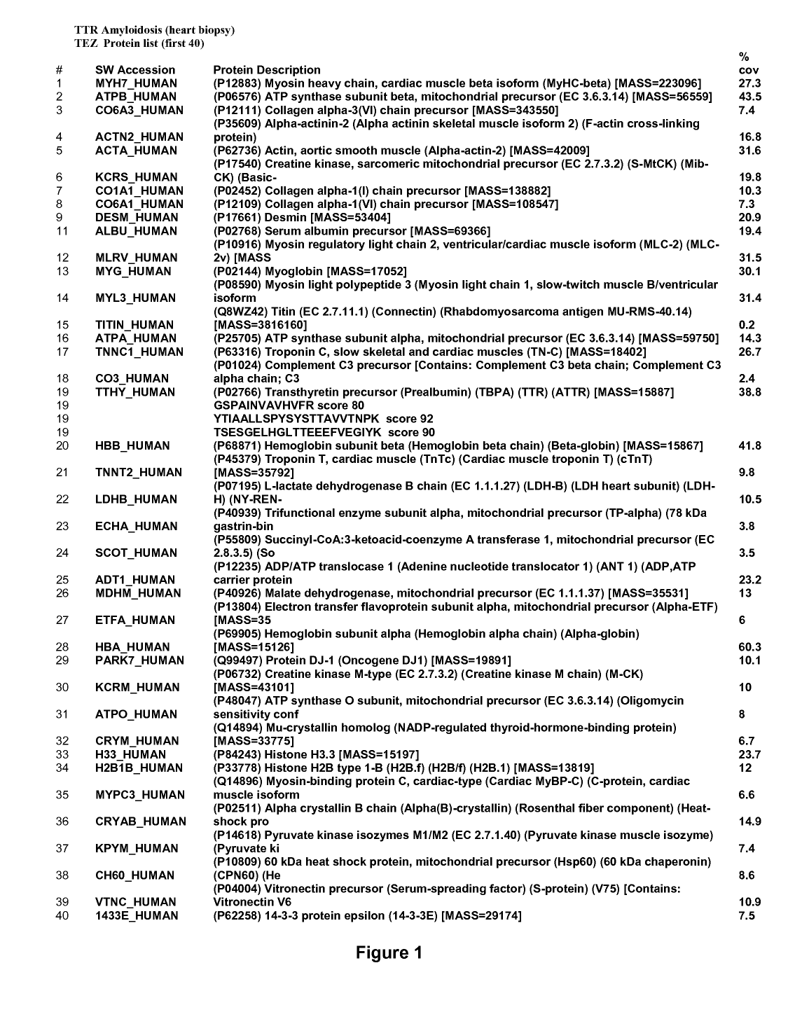

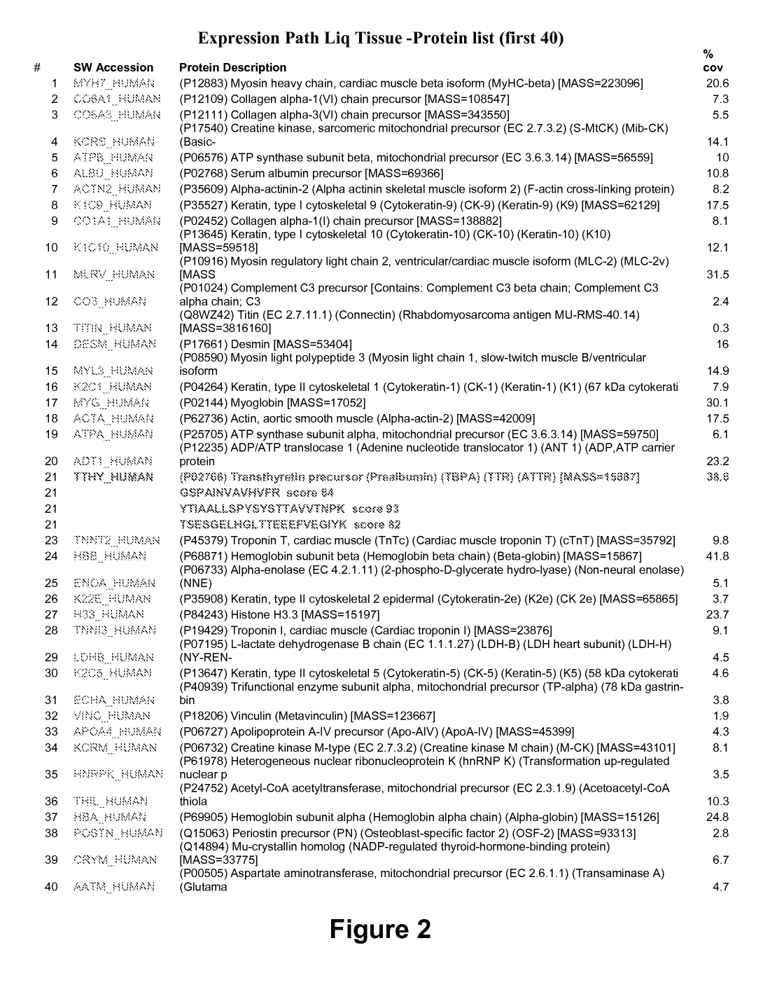

Two similar sized FFPE tissue samples were captured (˜50,000 μm²) for each case. One was prepared using the Expression Pathology method; and the other using the TEZ reagents. The top hits that had at least two unique peptides scoring 30 or higher are provided (FIGS. 1 and 2). No other amyloid polypeptides were matched for each case. Two control captures from normal heart tissue were treated in an identical manner.

In summary, about 50 FFPE tissue samples from heart, kidney, lymph nodes, brain, liver, lung, bone marrow, liver, and bladder, were examined where the amyloid subtype had been previously identified by traditional methods such as immunohistochemistry. All cases, where an amyloid polypeptide was identified by mass spectrometry, matched the previously diagnosed subtype as determined by IHC. No more than one amyloid polypeptide was detected in controls and there were no cases of more than one amyloid polypeptide being matched indicating excellent sensitivity and specificity.

### Example 3

### Laser Dissection and Identification of Amyloid Polypeptides

This study used FFPE tissues from four cases of amyloidosis, previously characterized as transthyretin (TTR), serum amyloid-associated protein (SAA), and immunoglobulin light chain lambda (IGL) and Kappa (IGK). Laser dissection and capture of amyloid plaques from sections of FFPE that were previously stained with Congo Red or Hematoxylin were performed. Polypeptides were extracted, digested with trypsin, and mass determined by MS/MS analysis (Thermo LTQ-Orbitrap), and peptides identified using Scaffold software (includes Mascot, X!Tandem, Sequest algorithims) and the SwissProt database.

### Results

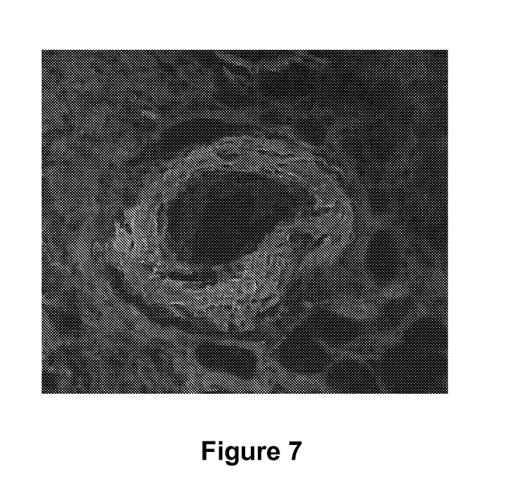

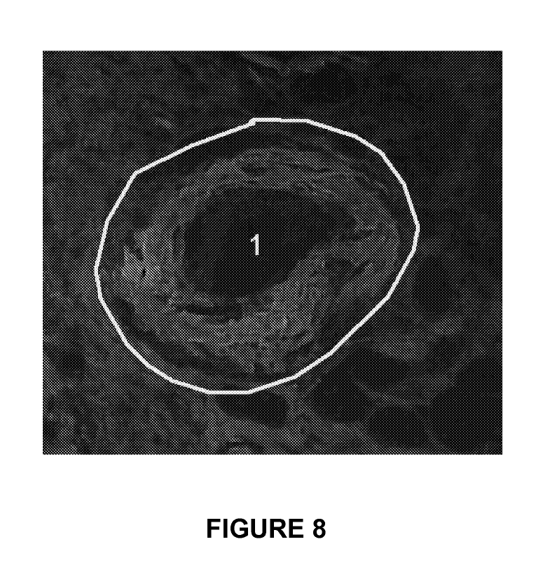

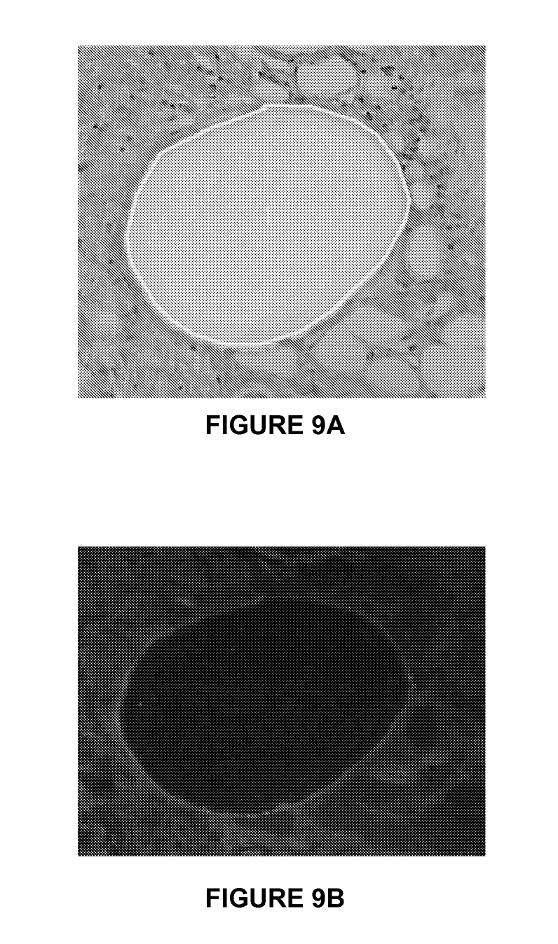

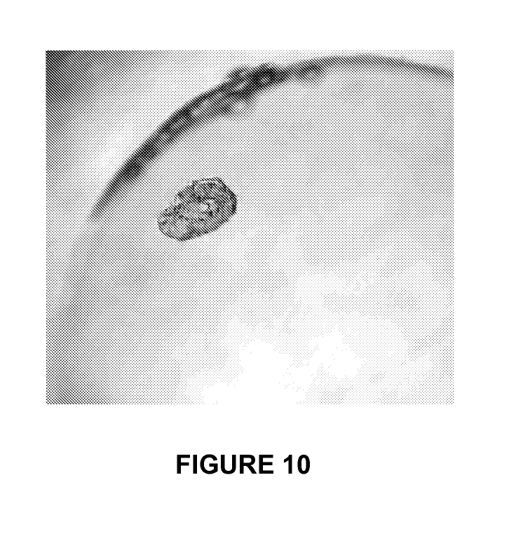

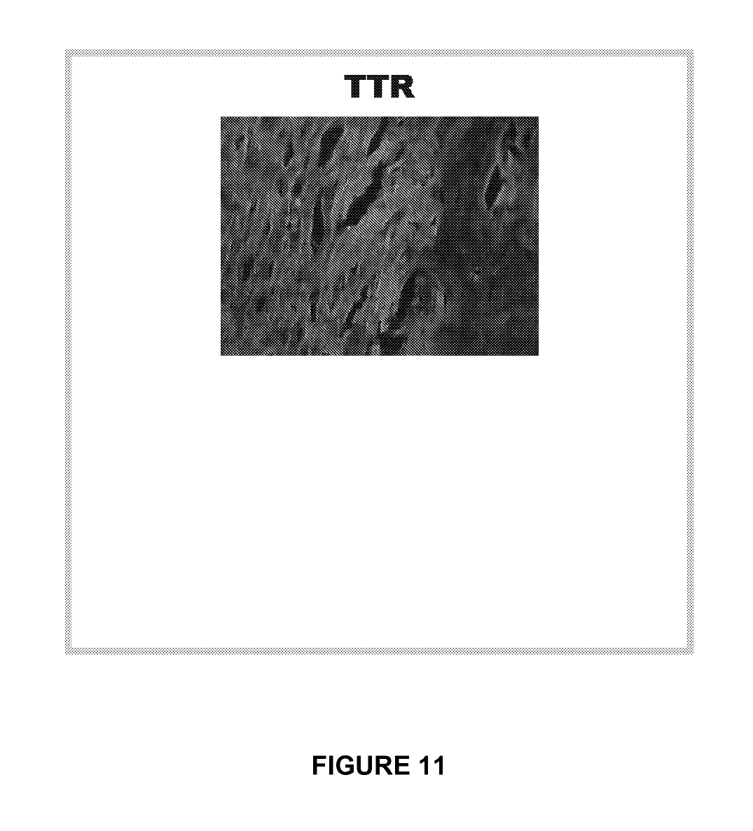

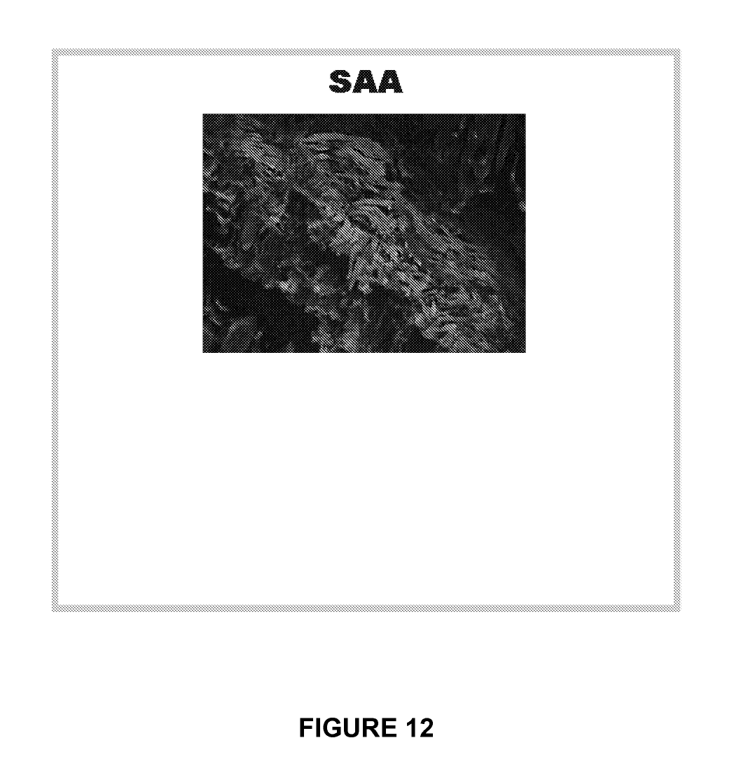

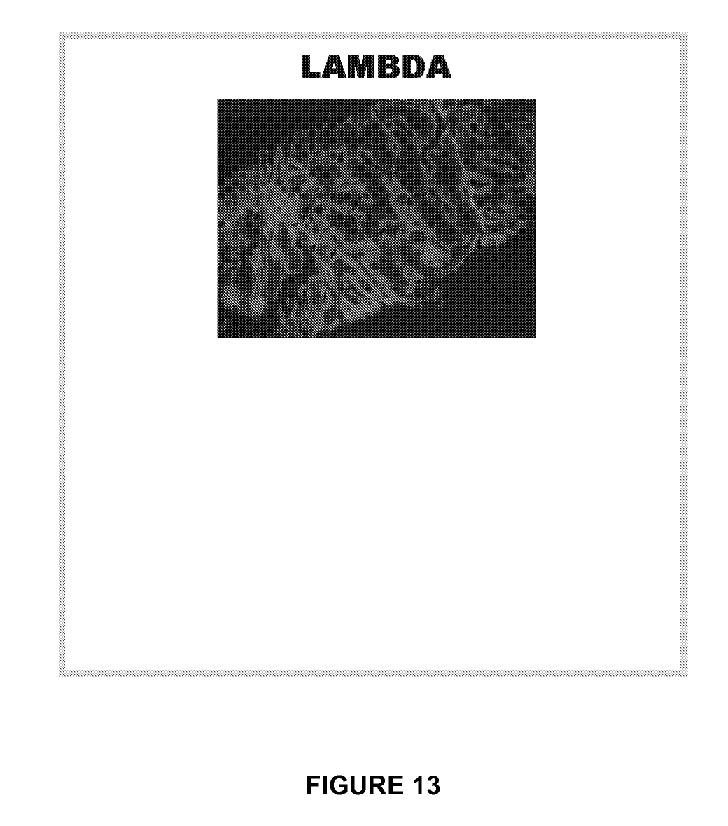

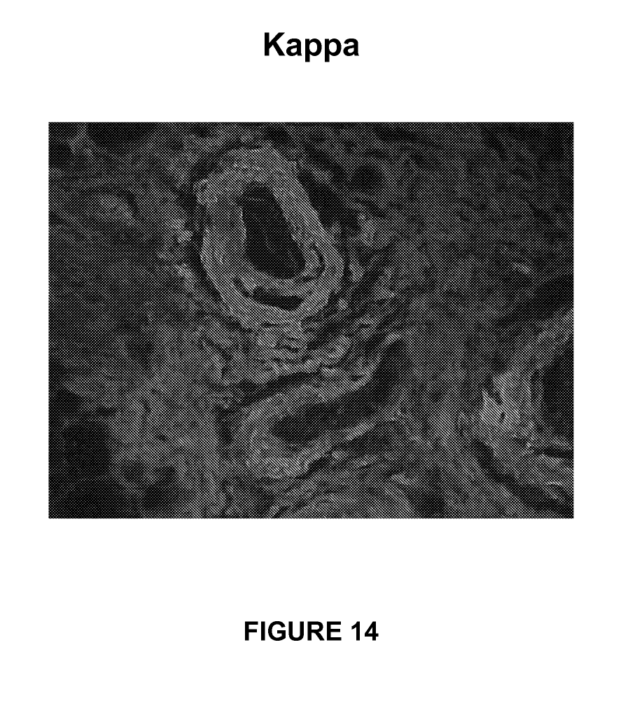

Mass spectrometry correctly identified each of the four types of amyloidosis analyzed with a high degree of specificity and sensitivity. In addition, peptide sequences containing mutations in the TTR polypeptide were identified that can allow further classification into senile or familial amyloidosis. Amino acid rearrangements in IGL and IGK were also seen. Additionally, Serum Amyloid P component (SAP) and Apoplipoprotein E were also identified as constituents of the amyloid deposition. These methods can be used as a clinical test for accurate identification of amyloid proteins in routinely processed biopsy specimens and can overcome many of the specificity and sensitivity issues associated with current methods such as immunohistochemistry of paraffin embedded sections. See, FIGS. 7-14.

The unique, targeted sampling ability of laser microdissection of Congo Red stained FFPE tissues, provides an amyloid enriched sample, which when combined with mass spectrometry based identification, provides a robust means for confident amyloid identification in clinical samples.

The use of laser microdissection from paraffin embedded biopsies and subsequent analysis by mass spectrometry allows identification of the type of amyloid polypeptide deposited with high specificity and sensitivity. Over 400 FFPE and 150 fat aspirate tissue samples have been analyzed using this methodology. This method can be a clinical test for accurate identification of amyloid polypeptides in routinely processed biopsy specimens and can overcome many of the specificity and sensitivity issues associated with current methods such as immunohistochemistry.

### Example 4

### Mass Spectrometry Analysis Identifies Two Distinct Types of Cutaneous Amyloidosis

Amyloid present in the skin can represent involvement by either a systemic disease or a primary, localized process. While the nonspecific amorphous deposits can appear the same by light microscopy, the underlying etiology can vary, and both keratins and immunoglobulin light chain can be implicated. As the management of amyloidosis targets underlying pathogenesis and may include high risk strategies, accurate classification is a clinical priority. To understand the pathogenesis of cutaneous amyloidosis, a proteomic approach involving microdissection and mass spectrometry based approaches was used and identified two distinct cutaneous amyloid types with characteristic clinico-pathological features.

Fifteen cases of cutaneous amyloidosis were identified from patient clinical records. In each case, the diagnosis of amyloidosis was confirmed by Congo red reactivity with appropriate color change under polarized light. Amyloid deposits were microdissected, processed, and trypsin digested into peptides. The peptides were analyzed by nano-flow liquid chromatography electrospray tandem mass spectrometry (LC-MS/MS). To identify the protein constituents of amyloid deposits, the resulting LC-MS/MS data were correlated to theoretical fragmentation patterns of tryptic peptide sequences from the Swissprot database using Scaffold program. To validate the findings of LC-MS/MS, immunohistochemistry for CK5, CK14, immunoglobulin kappa (IGK) and lambda light chains (IGL), TTR, and SAA was performed.

LC MS/MS identified two distinct protein profiles in cutaneous amyloid deposits. In ten cases, the amyloid deposits were enriched in cytokeratin 5 and 14 and, in the remaining five cases, peptides representing IGK (4/5) or IGL (1/5) were dominant. SAP was a constituent of both subsets. In each case, the findings of LC-MS/MS were confirmed by immunohistochemistry. Interestingly, the cases associated with CK5 and CK14 amyloid deposition were characterized by pruritis in the areas affected, with focal amyloid deposition at the dermal-epidermal junction. None of the patients had clinical evidence of systemic amyloidosis. In contrast, the cases with IGK or IGL deposition did not have an underlying inflammatory disorder, but three out of five had evidence for an underlying systemic plasma cell proliferative disorder. In these five cases, the amyloid deposits were more diffuse and involved deeper dermis as well as local vessels.

In summary, LC-MS/MS based proteomic analysis of cutaneous amyloidosis identified two distinct cutaneous amyloid types with characteristic clinico-pathological features. One was characterized by deposition of epidermis basal layer keratins, CK5, and CK14 probably due to increased basal cell damage and turnover caused by inflammatory dermatoses, while the other was characterized by deposition of IG light chains secondary to underlying plasma cell proliferative disorders. Recognition of these two distinct clinico-pathological types of cutaneous amyloidosis can be important to the clinical management of these patients. For example, patients found to have cutaneous amyloidosis characterized by deposition of epidermis basal layer keratins, CK5, and CK14 may not need treatment while patients found to have cutaneous amyloidosis characterized by deposition of IG light chains secondary to underlying plasma cell proliferative disorders may undergo clinical treatment.

### Example 5

### Diagnosis and Classification of Amyloidosis in Abdominal Subcutaneous Fat Aspiration Specimens Using Mass Spectrometry

Abdominal subcutaneous fat aspiration can be a practical, sensitive, and specific method for diagnosing systemic amyloidosis. A limitation of this method, compared to more invasive tissue biopsy based approaches, can involve the technical difficulties in further classification of the amyloidosis as commonly used methods such as immunohistochemistry may not be readily applicable to fat aspiration specimens. To overcome this, a method using nano-flow liquid chromatography electrospray tandem mass spectrometry (LC-MS/MS) was developed that can identify amyloid subtypes in freshly obtained Congo Red positive fat aspirate specimens with great accuracy.

Abdominal subcutaneous fat aspirate specimens were obtained from 73 patients with clinical suspicion for systemic amyloidosis. One half of each specimen was stained with Congo red and used for diagnosis of amyloidosis, and the other half of each specimen was processed and enzyme digested for LC-MS/MS analysis. The resulting LC-MS/MS data was correlated to theoretical fragmentation patterns of tryptic peptide sequences from the Swissprot database using Scaffold (Mascot, Sequest, and X!Tandem search algorithms). Peptide identifications were accepted if they could be established at greater than 90.0% probability, and protein identifications were accepted if they could be established at greater than 90.0% probability and contained at least two identified spectra. The identified proteins were subsequently examined for the presence or absence of amyloid related peptides. Of the 73 cases studied, 41 were positive for Congo red consistent with systemic amyloidosis. In Congo red positive cases, LC-MS/MS peptide profiles consistent with AL-lambda (28/31), AL-kappa (6/7), and ATTR (2/3) were observed. Only one case in the Congo red negative control group (31/32) gave a kappa light chain profile which was attributed to a high level of kappa in the serum (285 mg/dL). Of the 35 out of 41 cases of systemic amyloidosis successfully classified by LC-MS/MS, additional clinical and pathology data validating the amyloid type was available. In each of these cases, the MS/MS results accurately predicted the amyloid type.

In summary, LC-MS/MS analysis of abdominal subcutaneous fat aspiration specimens involved by amyloidosis provided a highly specific (97% specificity) and sensitive (>85% sensitivity) method for diagnosis and classification of amyloidosis. The method was rapid and is readily applicable in a clinical setting and can greatly improve the clinical management of amyloidosis patients.

### Example 6

### Immunoglobulin Light Chain Gene Constant Region is an Invariable Part of Amyloid Deposits in AL Amyloidosis

Amyloidosis caused by immunoglobulin light chain (IGLC) deposition, so-called AL-type or primary amyloidosis, is the most common type of amyloidosis. It has been long believed that IGLC variable regions form the core of the AL-type amyloid deposits and peptides derived from IGLC constant region peptides are only occasionally integrated into this core. For this reason, the scientific effort to identify the risk factors for development of AL amyloidosis and the biochemical characteristics of amyloid deposits has focused on IGLC variable region derived proteins. To understand the peptide constituents of AL amyloidosis better, a comprehensive study of AL amyloidosis was performed using a mass spectrometry based proteomic analysis approach.

Paraffin embedded tissue from 100 cases of AL amyloidosis was studied. In each case, amyloid type was previously established by clinical and pathological examination. Congo red stained paraffin sections were prepared, and amyloid deposits were microdissected by laser microdissection microscopy. The microdissected tissue fragments were processed and trypsin digested into peptides. The peptides were analyzed by nano-flow liquid chromatography electrospray tandem mass spectrometry (LC-MS/MS). The resulting LC-MS/MS data were correlated to theoretical fragmentation patterns of tryptic peptide sequences from the Swissprot database using Scaffold (Mascot, Sequest, and X! Tandem search algorithms). Peptide identifications were accepted if they could be established at greater than 90.0% probability, and protein identifications were accepted if they could be established at greater than 90.0% probability and contained at least two identified spectra. The identified proteins were subsequently examined for the presence or absence of amyloid related peptides.

LC-MS/MS gave peptide profiles consistent with AL amyloidosis in each case. The analysis revealed IGLC-lambda deposition in 66 cases and IGLC-kappa deposition in 34 of cases. In each case, LC MS/MS confirmed the previous clinicopathological diagnosis. Interestingly, peptides representing IGLC constant region were present in each case. Using this LC-MS/MS methodology, theoretically it is possible to cover 96% of the IGLC-lambda and 87% IGLC-kappa constant regions. For the tested samples, the average coverage of the IGLC-lambda and IGLC-kappa constant regions were 56% (range 14-78%) and 68% (range 16-87%), respectively. Additionally, the distribution of the peptides suggested that in the majority of the cases, the entire IGLC constant region was deposited. LC MS/MS also identified IGLC-lambda variable region peptides in 37 of 66 cases and IGLC-kappa variable region peptides in 29 of 34 cases studied. The variable region coverage was much more limited than the constant region coverage and included both framework areas and CDR regions. It is likely that the peptides derived from the variable region were present but not readily detected by the methodology as somatic hypermutation randomly alters the amino acid sequence in the CDR segments and such new sequences are not available in public databases used by algorithms for peptide identification. In the cases with the IGLC variable region hits, it was also possible to assign variable region family usage. IGLC-lambda cases frequently used IGLC-lambda variable region I, II, and III families whereas, in IGLC-kappa cases, IGLC-kappa variable region I and III families dominated.

In summary, AL amyloidosis can be accurately diagnosed using laser microdissection and LC-MS/MS based techniques in routine clinical specimens. In addition, AL amyloidosis invariably contains IGLC constant region peptides and, frequently, the whole of the constant region is deposited. These findings suggest that studies on molecular pathogenesis of amyloidosis should not only consider the IGLC-variable region but also the constant region. The results provided herein also indicate that it is possible to identify IGLC variable region family usage in AL amyloidosis using LC MS/MS based proteomic analysis. In the clinical setting, this information can be helpful in predicting organ distribution and clinical outcome.

### Example 7

### Diagnosis and Typing of Cardiac Amyloidosis in Routine Clinical Specimens by Mass Spectrometry Based Proteomic Analysis

Cardiac amyloidosis can be a frequent cause of restrictive cardiomyopathy and, if untreated, can lead to cardiac failure and death. Treatment strategies can target the underlying pathogenesis and often involve high risk approaches such as organ transplantation. Therefore, accurate typing of amyloid can be of clinical significance.

56 cases of paraffin embedded cardiac biopsies involved by amyloidosis and 4 cases of normal cardiac biopsies were studied. Congo red positive amyloid plaques were laser microdissected, trypsin digested, and analyzed by nano-flow liquid chromatography electrospray tandem MS (LC-MS/MS). The resulting LC-MS/MS data was correlated to theoretical fragmentation patterns of tryptic peptide sequences from the Swissprot database using Scaffold. Peptide identifications were accepted if established at greater than 90.0% probability, and protein identifications were accepted if established at greater than 90.0% probability and contained at least two identified spectra. The identified proteins were examined for the presence or absence of amyloid related peptides. Immunohistochemistry for immunoglobulin kappa (IGK) and lambda (IGL) light chains, transthyretin (TTR), serum amyloid A (SAA) was performed in 52 cases.

In 53/56 cases studied, LC MS/MS identified the presence of a single amyloidogenic protein. 35 cases exhibited a peptide profile consistent with TTR, 15 cases with IGL, 2 cases IGK, and 1 case with SAA. No amyloidogenic peptides were identified in normal cardiac stroma or muscle. Of the cases where immunohistochemistry was performed, staining was considered to be diagnostic in 19 cases and inconclusive in 33 cases. In each case, immunohistochemistry confirmed LC MS/MS findings. In those cases where immunohistochemistry was non-contributory, additional clinical and pathological information supported the amyloid type assigned by mass spectrometry.

In summary, LC-MS/MS proteomic analysis provided a highly specific and sensitive method for diagnosis and classification of amyloidosis in cardiac biopsy specimens. The method was rapid and readily applicable in a clinical setting to paraffin embedded tissues and can improve the diagnosis and clinical management of cardiac amyloidosis.

### Example 8

### Leukocyte Chemotactic Factor 2 Amyloidosis: A Type of Amyloidosis that Mimics AL Amyloidosis

Amyloidosis is caused by an abnormal extracellular deposition of serum proteins in a beta pleated sheet structure that disrupts normal biological functions of vital organs. The most common causes of systemic amyloidosis include the immunoglobulin light-chain (AL), hereditary or senile transthyretin (ATTR), and serum amyloid associated protein (AA) amyloidosis. In a small subset of amyloidosis, however, no cause can be identified either by pathological or clinical investigations. In this study, a novel-type of amyloidosis caused by deposition of a chemokine, leukocyte chemotactic factor 2 (LECT2), is described. This amyloidosis may account for the majority of unclassifiable amyloidoses.

Seven cases of amyloidosis, where amyloid type can not be determined after extensive pathological, laboratory, and clinical investigations, were studied (Table 1). In each case, the diagnosis of amyloidosis was made on histological examination of a tissue biopsy specimen and positive Congo red staining. The amyloid typing was performed by nano-flow liquid chromatography electrospray tandem mass spectrometry (MS/MS) following laser microdissection and proteolytic digestion of the amyloid deposits. The resulting MS/MS data was correlated to theoretical fragmentation patterns of tryptic peptide sequences from the Swissprot database using Scaffold algorithm. Peptide identifications were accepted if they could be established at greater than 90.0% probability, and protein identifications were accepted if they could be established at greater than 90.0% probability and contain at least two identified spectra. The identified proteins were subsequently examined for the presence or absence of amyloid related peptides. Additionally, immunohistochemistry was performed for SAA, IGK, IGL, TTR, and LECT2 proteins. 40 cases of AL amyloidosis, 20 cases of TTR amyloidosis, and 10 cases of renal glomeruli not involved by amyloidosis were analyzed as controls.

Demographic and clinical features of all cases studied were summarized (Table 1). In each case, MS/MS analysis revealed that one of the most abundant peptides was LECT2. No other amyloid associated peptides were present. In contrast, in the control cases involved by AL or ATTR, no LECT2 peptides were present. Similarly, the amyloid negative renal control specimens did not contain LECT2 peptides indicating that the findings were specific. Immunohistochemical studies confirmed the findings of MS/MS.

TABLE 1

| | | | Organ | |
|---|---|---|---|---|
| Case No. | Age/Sex | Presentation | involvement | Other history |
| 1 | 69 M | Nephrotic syndrome | Kidney | Siblings with |
| | | | | renal disease |
| 2 | 75 M | Nephrotic syndrome | Kidney | Siblings with |
| | | | | renal disease |
| 3 | 76 M | Nephrotic syndrome | Kidney | |
| 4 | 59 F | Nephrotic syndrome | Kidney | |
| 5 | 63 M | Nephrotic syndrome | Kidney | |
| 6 | 55 M | Chronic hepatitis B | Liver | |
| 7 | 76 F | Incidental during | Liver, gall | |
| | | cholecystectomy | bladder | |

These results demonstrate the existence of a novel-type of amyloidosis caused by deposition of LECT2. LECT2 amyloidosis typically presents with isolated renal involvement and nephrotic syndrome but liver involvement may also be seen. The clinical and pathological features of LECT2 amyloidosis closely mimic AL amyloidosis and should be considered in the differential diagnosis. The underlying cause for LECT2 amyloidosis remains unknown.

### Example 9

### Immunoglobulin Derived Depositions in the Nervous System

### Patients

Pathology records were searched for cases of amyloid and other proteinaceous material deposition (non-amyloid extracellular deposits and intracellular crystals) in brain, spinal cord, or peripheral nerve. Thirteen (of 15) cases were included in this study. Two cases were excluded because only unstained charged slides were available for analysis. Twelve of the thirteen cases were biopsies, and one case was obtained from autopsy (case 9). All slides were reviewed by at least two neuropathologists and one hematopathologist. Clinical data, demographics, and follow-up information were obtained from retrospective chart review and consultation correspondence.

### Specimen Preparation and Microdissection

The duration of storage for the formalin-fixed paraffin-embedded (FFPE) tissues used in this study was variable, with a range of 6 months to 10 years. Thick (10μ) sections were placed on DIRECTOR™ slides (Expression Pathology, Gaithersburg, Md.) for ease of laser microdissection. The tissue was dissected directly from a charged slide in cases were paraffin blocks were not available. At least three different areas were separately microdissected and analyzed in all cases. Sections were air dried then melted, deparaffinized, and stained in hematoxylin followed by Congo Red. Fluorescence microscope optics was used to identify areas of Congo Red (CR) positivity. In the non-amyloid cases, the areas of interest were microdissected under bright-field microscopy into 0.5 mL microcentrifuge tube caps containing 10 mM Tris/1 mM EDTA/0.002% Zwittergent 3-16 (Calbiochem, San Diego Calif.) using a Leica DM6000B Microdissection System (Wetzler, Germany). Collected tissues were heated at 98° C. for 90 minutes with occasional vortexing. Following 60 minutes of sonication in a waterbath, samples were digested overnight at 37° with 1.5 μL of 1 μg/mL trypsin (Promega, Madison, Wis.).

### Protein Identification Via Mass Spectrometry

The trypsin generated digests were reduced with dithiothreitol (DTT) and separated by nano-flow liquid chromatography electrospray tandem mass spectrometry (nanoLC-ESI-MS/MS) using a ThermoFinnigan LTQ Orbitrap Hybrid Mass Spectrometer (ThermoElectron Bremen, Germany) coupled to an Eksigent nanoLC-2D HPLC system (Eksigent, Dublin, Calif.). A 0.25 μL trap (Optimize Technologies) packed with Michrom Magic C-8 was plumbed into a 10-port valve. A 75 μm×˜15 cm C-18 column was utilized for the separation utilizing an organic gradient from 6% to 86% in 55 minutes at 400 nL/min.

The Thermo-Fisher MS/MS raw data files were submitted to an in-house developed workflow tool (Lentz et al., Proteome Workflow: Workflow Tool for Building Proteomics Workflows, in 55th Annual American Society for Mass Spectrometry. Indianapolis, Ind. USA, American Society for Mass Spectrometry, 2007). Three search algorithms (Sequest, Mascot, and X!Tandem) were searched, and the results assigned peptide and protein probability scores. The results were then displayed in Scaffold (Proteome Software, Portland Oreg.). All searches were conducted with variable modifications and restricted to full trypsin generated peptides allowing for two missed cleavages. Peptide mass search tolerances were set to 10 ppm, and fragment mass tolerance to ±1.00 Daltons. The human SwissProt database was utilized. Protein identifications were confirmed at the 90% confidence level and required two peptides for identification.

### Immunohistochemistry

Immunohistochemical stains were performed with the aid of a DAKO Autostainer (Dako North America, Inc., Carpinteria, Calif.) using the Dual Link Envision+ or ADVANCE (Dako) detection systems. Antibodies were directed against the following antigens, with the corresponding clones for the monoclonal antibodies specified: CD3 (Novocastra, Newcastke, UK; clone PS1; dilution 1:50), CD20 (Dako; clone L26; dilution 1:60), CD68 (Dako, clone KP-1; dilution 1:3000), CD138 (Dako; clone M115; dilution 1:50), Kappa Free Light Chains (Dako, polyclonal, dilution 1:6000), Kappa Light Chain (Auto ProEnzyme pretreatment; Dako; polyclonal; dilution 1:2500), Lambda Free Light Chains (Dako; polyclonal; dilution 1:2000), Lambda Light Chain (Auto ProEnzyme pretreatment; Dako; polyclonal; dilution 1:3000), Prealbumin (Transthyretin)(Dako; polyclonal; dilution 1:5000), Serum Amyloid A (SAA)(Dako; clone MC-1; dilution 1:1000), and Serum Amyloid P (SAP)(Biocare; polyclonal, dilution 1:20).

### In Situ Hybridization

In situ hybridization with probes targeting Lambda and Kappa light chain mRNA was performed on case 7 using Ventana Benchmark XT platform (Ventana, Tucson, Ariz.) and commercially available probes (Inform® cytoplasmic kappa and lambda probes, Ventana) according to the manufacturer's instructions.

### Results

### Clinical Findings

The clinical findings were summarized in Table 2. The depositions involved brain (n=12) or peripheral nerve (n=1). The patients included 9 women and 4 men. They were all adults with a median age at diagnosis of 51 years (range 31-72). Radiologic studies demonstrated multifocal lesions in the majority of the cases, usually involving cerebral white matter (n=6). A single abnormality/mass was present in 5 cases. In the single case involving peripheral nerve, a localized enhancing enlargement of the sciatic nerve was evident. No imaging data was available for the remaining case.

TABLE 2
Protein Deposits in Clinical Specimens of the Nervous System: Clinical
Features.

| | | Clinical | | | | | Systemic | Follow- |
|---|---|---|---|---|---|---|---|---|
| Patient | Age/sex | History | Imaging | Location | Surgery | Treatment | work-up | up |
| 1 | 2/F | Headache, | R parietal | Bilateral, | L temporal | Radiation | Bone Marrow | Recovered |
| | | dizziness, and | lobe: ring | multifocal | craniotomy | | biopsy negative | well after |
| | | gait problems | enhancing | | with biopsy | | Blood and urine | treatment. |
| | | for several | mass (3.6 cm), | | | | elecrophoresis | Lost to |
| | | months; brain | moderate | | | | negative | follow-up. |
| | | lesions may | surrounding | | | | | |
| | | have been | white matter | | | | | |
| | | present for 20 | edema, mass | | | | | |
| | | years | effect. | | | | | |
| | | | L temporal | | | | | |
| | | | lobe: | | | | | |
| | | | confluent | | | | | |
| | | | enhancing | | | | | |
| | | | masses (4.2 cm), | | | | | |
| | | | surrounding | | | | | |
| | | | edema; | | | | | |
| | | | increased T1 | | | | | |
| | | | signal, | | | | | |
| | | | decreased T2 | | | | | |
| | | | signal in the | | | | | |
| | | | center; stable | | | | | |
| | | | over several | | | | | |
| | | | months | | | | | |
| 2 | 5/M | Sudden onset | MRI: | L | Stereotactic | Observation with | Chest CT- | Developed |
| | | of | enhancing 1.1 cm | posterolateral | biopsy | yearly MRIs. | negative for | pulmonary |
| | | hemiparesis; | lesion; | thalamus/internal | | | lesions. | embolism two |
| | | received | gradual | capsule | | | Systemic work- | months after |
| | | steroids | enlargement | | | | up not done. | surgery. |
| | | followed by | with ring | | | | | Stable lesion |
| | | biopsy | enhancement | | | | | on yearly |
| | | | development | | | | | MRIs two |
| | | | | | | | | years after |
| | | | | | | | | surgery. |
| 3 | 0/F | Seizures | Well | Brain NOS | Biopsy | NA | NA | Lost to |
| | | | demarcated | | | | | follow-up |
| | | | contrast | | | | | |
| | | | enhancing, | | | | | |
| | | | hemispheric | | | | | |
| | | | lesion | | | | | |
| 4 | 1/M | Left | MRI: ring | R occipital | Stereotactic | Observation | Observation | Persistent |
| | | homonymous | enhancing | lobe | biopsy | followed by | with MRIs. | symptoms |
| | | inferior | mass | | | resection | Followed by | slight |
| | | quadrantonopsia | | | | | Neurosurgery | enlargement |
| | | | | | | | only. | on follow-up |
| | | | | | | | | MRI. |
| | | | | | | | | Underwent |
| | | | | | | | | resection 1 |
| | | | | | | | | month after |
| | | | | | | | | which |
| | | | | | | | | revealed |
| | | | | | | | | amorphous |
| | | | | | | | | debris with a |
| | | | | | | | | mild benign |
| | | | | | | | | polyclonal |
| | | | | | | | | lymphoplasma |
| | | | | | | | | cytic infiltrate. |
| | | | | | | | | No evidence |
| | | | | | | | | of recurrence |
| | | | | | | | | after (most |
| | | | | | | | | recent MRI 3 |
| | | | | | | | | years after |
| | | | | | | | | diagnosis). |
| 5 | 4/F | Problems with | Periventricular, | Bilateral | Stereotactic | Radiation therapy | Negative fat | Stable, |
| | | balance 5 | nodular, | multifocal | right frontal | | aspirate | neurologically |
| | | years; Gait | perivascular, | | biopsy | | CSF normal | and by |
| | | disturbance | enhancing | | | | Normal serum | imaging 8 |
| | | most apparent | white matter | | | | and urine | months post- |
| | | 19 months | lesions in | | | | electrophoresis | biopsy; stable |
| | | prior | frontoparietal | | | | | neurologically |
| | | | lobes, R > L, | | | | | one and a half |
| | | | progressive in | | | | | months post- |
| | | | 8 months | | | | | biopsy |
| 6 | 9/M | History of | Multiple white | Bilateral | L frontal lobe | Treated with | Negative CT | Neurologically |
| | | Lhermitte sign | matter | periventricular | and dural | methotrexate and | chest and | stable 14 |
| | | and optic | enhancing | white matter | biopsy | fludarabine with | abdomen | months post- |
| | | neuritis. | lesions, some | | | partial response. | | biopsy |
| | | Diagnosed | periventricular, | | | | | Family history |
| | | with multiple | with | | | | | of Alexander |
| | | sclerosis | dominant 3 cm | | | | | disease |
| | | | L frontal | | | | | |
| | | | mass | | | | | |
| 7 | 1/F | Recurrent | Multiple | Bilateral white | Stereotactic | Steroids | Negative bone | Slight |
| | | spells of | enhancing | matter | biopsies × 2 | | marrow biopsy, | improvement |
| | | aphasia and | lesions, | | | | fat aspirate. | in imaging |
| | | right sided | predominantly | | | | Negative Body | findings 17 |
| | | weakness for | white matter, | | | | CT and MRI of | months post- |
| | | 2 years | involving left | | | | spine. | biopsy |
| | | | hemisphere | | | | Normal serum | |
| | | | and | | | | and urine | |
| | | | cerebellum | | | | electrophoresis | |
| | | | | | | | Elevated | |
| | | | | | | | kappa/lambda | |
| | | | | | | | ratio in CSF | |
| | | | | | | | (3.79). | |
| 8 | 0/F | Transient R | “L temporal | Bilateral, | L temporal | Alkeran and | Normal bone | Persistent |
| | | sided | lobe AVM” | multifocal | lobe | prednisone × 5 | marrow biopsy | seizure |
| | | weakness 3 | Stellate | | stereotactic | cycles | and fat aspirate | disorder; |
| | | years prior; | enhancement | | biopsy | | Normal serum | stable lesions |
| | | multifocal | and T2 | | | | and urine | on subsequent |
| | | muscle aches | abnormality in | | | | electrophoresis | studies but |
| | | of unclear | deep white | | | | | most recent |
| | | etiology | matter of R | | | | | MRI 9 years |
| | | | frontal lobe | | | | | after first |
| | | | | | | | | biopsy shows |
| | | | | | | | | new lesions of |
| | | | | | | | | potential |
| | | | | | | | | concern. |
| 9 | 8/F | Weakness, | MRI: | Bilateral white | 1-R frontal | Rituximab, high | CT of | DOD 30 days |
| | | nausea, | multiple, | matter | lobe biopsy | dose steroids, | abdomen: | after onset of |
| | | vomiting, | ovoid, non- | | 2-Autopsy | plasmapharesis | splenomegaly | symptoms |
| | | changes in | enhancing | | | | Serum | |
| | | mental status | lesions in | | | | electrophoresis: | |
| | | | cerebral and | | | | monoclonal | |
| | | | cerebellar | | | | kappa light | |
| | | | white matter | | | | chains | |
| | | | | | | | Bone marrow | |
| | | | | | | | biopsy: B-cell | |
| | | | | | | | lymphoma | |
| | | | | | | | Autopsy: | |
| | | | | | | | Splenic | |
| | | | | | | | Marginal Zone | |
| | | | | | | | Lymphoma | |
| 10 | 2/F | Tinnitus | CT: densely | R parietal lobe | biopsy | NA | NA | Lost to |
| | | | enhancing | | | | | follow-up |
| | | | mass lesion | | | | | |
| | | | deep in the | | | | | |
| | | | white matter | | | | | |
| | | | adjacent to the | | | | | |
| | | | occipital horn | | | | | |
| | | | of the R | | | | | |
| | | | ventricle | | | | | |
| 11 | 0/F | NA | NA | R parietal lobe | biospy | NA | NA | Neurologically |
| | | | | | | | | stable 3 |
| | | | | | | | | months post- |
| | | | | | | | | biopsy |
| 12 | 2/M | L leg | MRI: | S1, S2 nerve | Fascicular | Observation | Bone marrow | Stable 1 year |
| | | weakness and | enlargement | roots | biopsy | | biopsy, fat | post-biopsy |
| | | paresthesias | through time | extending into | | | aspirate, and | |
| | | starting | with mild | sciatic nerve | | | free light chains | |
| | | approximately | enhancement | | | | in serum and | |
| | | 8 years prior | | | | | urine negative. | |
| 13 | 4/F | “Stroke” | MRI: “tumor” | L frontal dura | Resection, | NA | NA | Developed |
| | | followed by | | | followed by | | | nodular |
| | | seizure 1 | | | open biopsies | | | enhancement |
| | | month prior | | | 3 and 12 | | | in right |
| | | | | | months after | | | parietal and |
| | | | | | | | | occipital |
| | | | | | | | | (extraaxial) |
| | | | | | | | | regions |
| | | | | | | | | prompting the |
| | | | | | | | | third biopsy |

NA = data not available,
MRI = magnetic resonance imaging,
CT = computed tomography,
L = left,
R = right,
AVM = arteriovenous malformation,
DOD = dead of disease

### Pathology

Histologically the proteinaceous deposits could be placed within one of the following categories: extracellular proteinaceous deposit not otherwise specified (PDNOS) (n=6), amyloidoma (n=5), or intracellular crystals (n=2). The pathologic features are summarized in Table 3.

TABLE 3
Protein Deposits in Clinical Specimens of the Nervous System: pathologic
features.

| Patient | Diagnosis | Histopathology | Congo Red | Immunohistochemistry | Mass Spectrometry |
|---|---|---|---|---|---|
| 1 | Amyloidoma | Eosinophilic | Positive | Lambda light chain positivity in | Lambda, ApoE, Apo-AIV, and |
| | | parenchymal and vascular | | deposits and plasma cells | SAP peptides |
| | | aggregates with focal | | SAP+ | |
| | | giant cell reaction | | SAA−, Transthyretin−, Beta | |
| | | associated with | | microglobulin- | |
| | | perivascular plasma cell | | | |
| | | proliferation | | | |
| 2 | Neoplasm with | Prominent | Negative | Perivascular mixture of CD3 and | IgG peptide. |
| | extensive | intracytoplasmic | | CD20+ T and B cells CD138+ plasma | |
| | plasma cell | immunoglobulin | | cells, lambda Ig light chain. Crystals | |
| | differentiation | rhomboid/rectangular | | IgG+ | |
| | | crystals in plasma cells; | | | |
| | | rare touton like | | | |
| | | multinucleated giant | | | |
| | | cells; perivascular | | | |
| | | inflammation | | | |
| 3 | Low grade B- | Flocculent extracellular | Negative | Kappa light chain restricted | Kappa, IgG, and ApoE |
| | cell lymphoma | proteinaceous deposit | | lymphoplasmacytic cells. | peptides |
| | with | with occasional giant cell | | Predominant CD20+ B-cells with a | |
| | plasmacytic | reaction, | | minor CD138+plasma cell component | |
| | differentiation | lymphoplasmacytic | | | |
| | | infiltrate mainly | | | |
| | | perivascular | | | |
| 4 | 1-Amorphous | Amorphous eosinophilic | Negative | inconclusive | Kappa, IgG and IgA peptides. |
| | debris | debris with dystrophic | | | |
| | 2-Old | calcifications, focal | | | |
| | organized | crystalline appearance, no | | | |
| | intraparenchym | inflammation | | | |
| | al cystic lesion | | | | |
| | with | | | | |
| | proteinaceous | | | | |
| | content | | | | |
| 5 | Amyloidoma | Parenchymal and | Positive | Lambda+ Aggregates, | Lambda, ApoE, Apo-AIV and |
| | | perivascular involvement; | | parenchymal and vessel walls; | SAP peptides |
| | | Scant perivascular | | SAP focal+ | |
| | | lymphoplasmacytic | | SAA−, beta microglobulin−, beta | |
| | | infiltrate | | amyloid− | |
| 6 | Extranodal | Granulomatous reaction | Negative | Kappa restricted B-cells, plasma cells | Kappa and IgG peptides |
| | Marginal Zone | to extracellular crystalline | | and extracellular crystals; IgM+cells, | |
| | Lymphoma | immunoglobulin | | aggregates−; IgA−, IgG non | |
| | | | | contributory. | |
| 7 | Atypical | Histiocytes engorged | ND | Small groups of kappa restricted | Kappa and GFAP peptides. |
| | plasma cell | with crytstals, scant | | plasma cells by in situ hybridization | |
| | infiltrate | perivascular plasma cells | | Cells with crystals are | |
| | suspicious for | | | CD68+histiocytes | |
| | plasma cell | | | Immunostains for kappa and lambda | |
| | proliferative | | | non contributory | |
| | disorder and | | | | |
| | crystal storing | | | | |
| | histiocytosis | | | | |
| 8 | Amyloidoma | Eosinophilic | Positive | Lambda+ | Lambda, SAP, ApoE, Apo-AI |
| | | parenchymal and | | Rare CD20+B-cells and CD138+ | and Apo-AIV peptides |
| | | vascular aggregates; | | plasma cells | |
| | | reactive gliosis | | | |
| 9 | Extracellular | Large eosinophilic | Negative | Kappa+, IgM+ | Kappa, IgM, Apo-AI, and IgJ |
| | proteinaceous | proteinaceous aggregates; | | IgA−, IgG− | peptides |
| | deposits in | no inflammation | | | |
| | brain; marginal | | | | |
| | zone lymphoma | | | | |
| | of spleen | | | | |
| 10 | Amyloidoma | Parenchymal and | Positive | Lambda free light chain +; | Lambda, SAP, IgG, IgA, |
| | | perivascular, gliosis, no | | kappa −, SAA−, prealbumin−, SAP− | ApoE, and Apo-AIV, and |
| | | inflammation | | | Apo-AI peptides |
| 11 | Proteinaceous | Extracellular eosinophilic | Negative | Kappa+ deposition, IgA, IgG and IgM | Kappa, IgA, IgG, Apo-AI, |
| | deposition | proteinaceous pools | | equivocal | Apo-AII and IgJ peptides |
| | | surrounded by | | | |
| | | lymphoplasmacytic | | | |
| | | infiltrate with | | | |
| | | eosinophils, collagen | | | |
| | | deposition | | | |
| 12 | Amyloidoma | Tumefactive mass of | Positive | Lambda+ | Lambda, SAP, ApoE, Apo-AI, |
| | | amyloid replacing a nerve | | | and Apo-AIV peptides |
| | | fascicle. | | | |
| | | Mild perivascular | | | |
| | | lymphocytic infiltrate in | | | |
| | | adjacent tissue | | | |
| 13 | Chronic | Mixed | Negative | Polytypic plasma cells | Lambda, Kappa, ApoE, IgG, |
| | inflammation | lymphoplasmacytic | | | IgM, C9, ApoA1, Ig heavy |
| | with | infiltrate with | | | chain. |
| | eosinophils | eosinophils, eosinophilic | | | |
| | | amorphous material, | | | |
| | | fibrosis | | | |

NA information not available;
ND not done;
SAP serum amyloid protein;
Ig Immunoglobululin

### Extracellular Proteinaceous Deposit Not Otherwise Specified

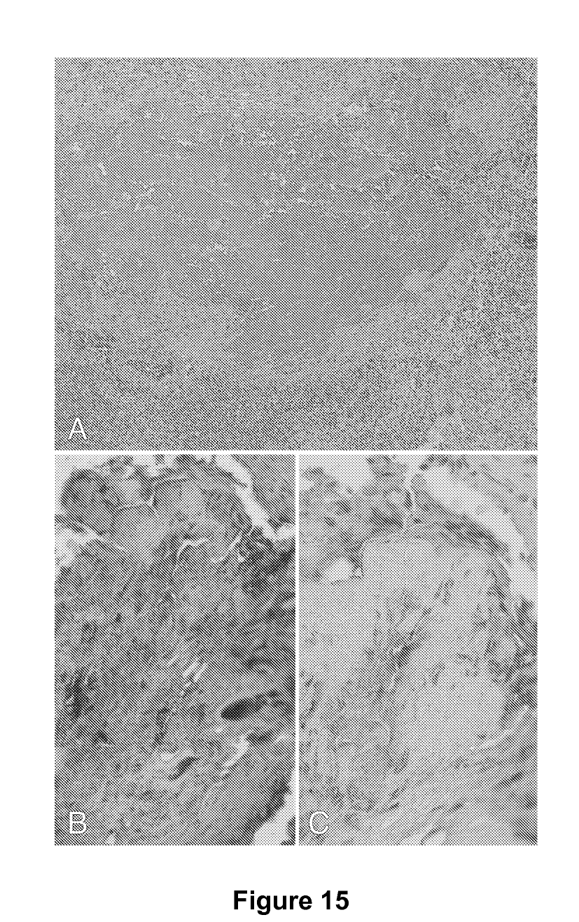

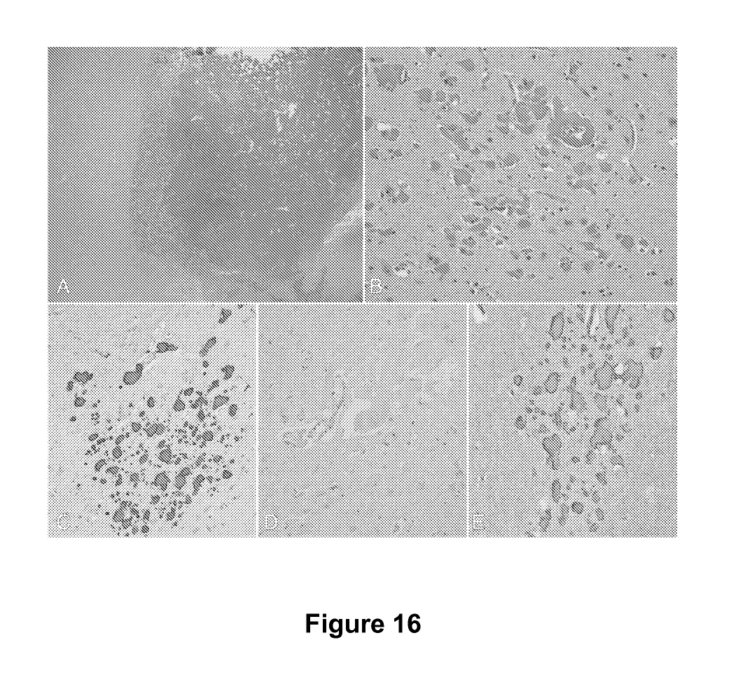

These consisted of amorphous, flocculent, Congo Red negative extracellular eosinophilic proteinaceous aggregates with occasional calcification. In two cases, well formed extracellular crystals were also present. Two cases were associated with a contiguous low grade B-cell lymphoma with lymphoplasmacytic differentiation and demonstrated a foreign body giant cell reaction to the deposits (FIG. 15). A marginal zone lymphoma of the spleen was identified at autopsy in one dramatic case characterized by deeply eosinophilic immunoglobulin lakes mostly in white matter but with smaller aggregates in the cortex (FIG. 16).

### Amyloidoma

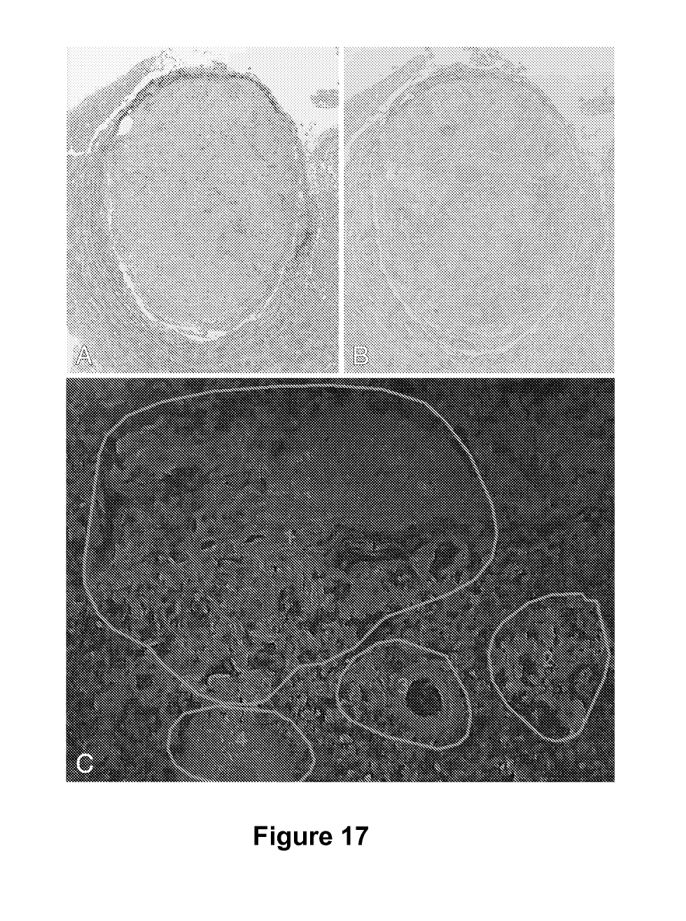

Amorphous pink aggregates of amyloid were present in a parenchymal and/or perivascular pattern (FIG. 17A). Congo red staining was positive in all cases, showing strong red fluorescence (FIGS. 17B and 17C). An associated mild, generally perivascular lymphoplasmacytic infiltrate was also identified.

### Intracellular Crystals

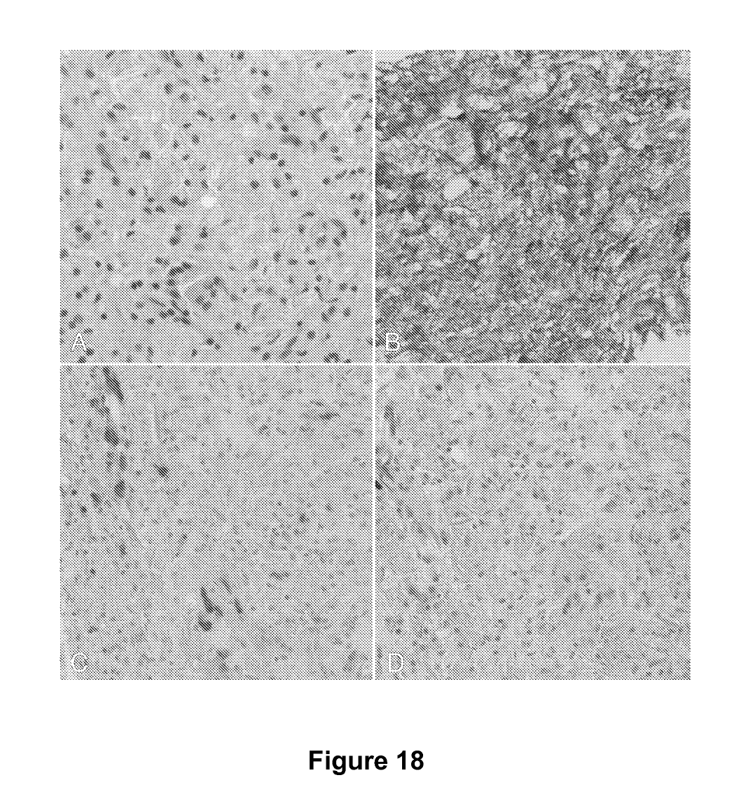

Intracellular crystals were the hallmark in two cases. In case 7, there was a massive intracellular accumulation of needle shaped to rhomboid crystals within CD68 positive macrophages throughout the biopsy consistent with intracerebral crystal storing histiocytosis (FIG. 18). Scattered, mostly perivascular plasma cells were present. In case 2, thicker rhomboid crystals were noted within CD138 positive plasma cells, which were less frequent than in case 7. “Touton-like” multinucleated giant cells were also present.

### Mass Spectrometry and Immunohistochemistry

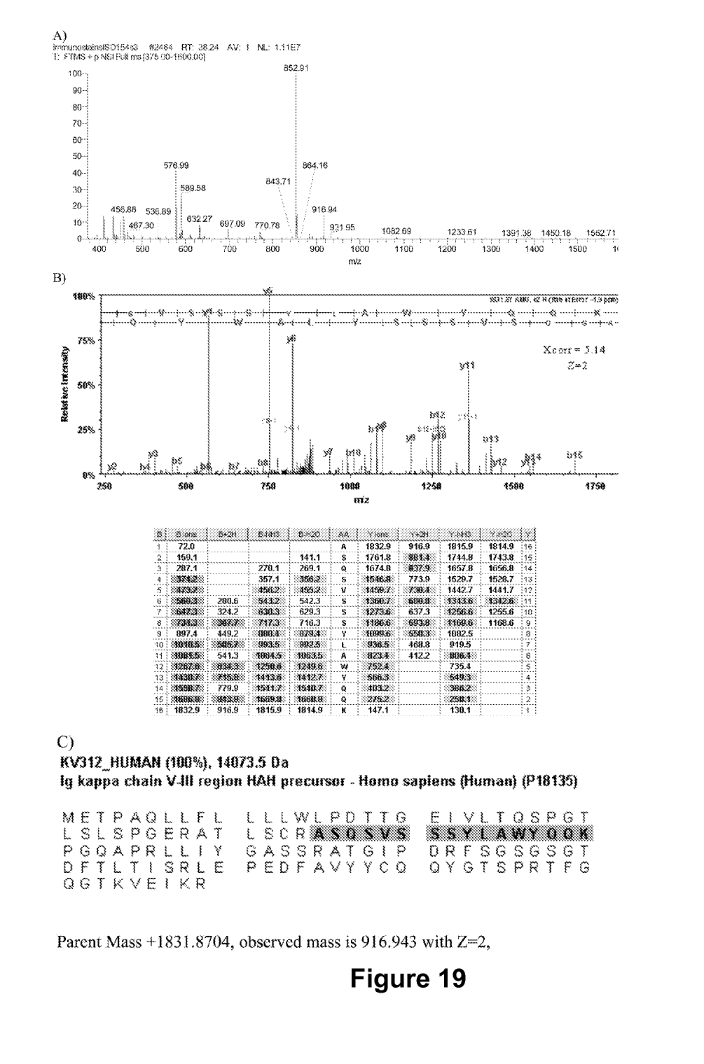

Proteomic analyses provided reliable MS and MS/MS spectra in a representative case (FIG. 19), and the Scaffold™ search algorithm provided reproducible protein profiles in all 13 cases tested (Tables 4-15).

TABLE 4
Scaffold Results for MS data case 1 with top 10 proteins identified by
probability based algorithm per case.

| | | | Molecular | | |
|---|---|---|---|---|---|
| | | | weight | Immunostains | Immunostains |
| | Identified Proteins | Accession number | (kDa) | SPA706305.mgf | SPA706306.mgf |
| 1 | Apolipoprotein E | APOE_HUMAN | 36 | 100% | 100% |
| | precursor | | | | |
| 2 | Ig lambda chain V- | LV302_HUMAN | 12 | 100% | 100% |
| | III region LOI | | | | |
| 3 | Serum amyloid P- | SAMP_HUMAN | 25 | 100% | 100% |
| | component | | | | |
| | precursor | | | | |
| 4 | Apolipoprotein A- | APOA4_HUMAN | 45 | 94% | 100% |
| | IV precursor | | | | |
| 5 | Serum albumin | ALBU-HUMAN | 69 | 100% | 100% |
| | precursor | | | | |
| 6 | Vitronectin | VTNC_HUMAN | 54 | 100% | 94% |
| | precursor | | | | |
| 7 | Collagen alpha-1(I) | CO1A1_HUMAN | 139 | 94% | 100% |
| | chain precursor | | | | |
| 8 | Multiple epidermal | MEGF8_HUMAN | 255 | 87 | 100 |
| | growth factor- | | | | |
| | ligand | | | | |
| 9 | Metallothionein-4 | MT4_HUMAN | 6 | 100 | 71 |
| 10 | Plasminogen | PLMN_HUMAN | 91 | 100 | |
| | precursor | | | | |

TABLE 5
Scaffold Results for MS data case 2 with top 10 proteins identified by
probability based algorithm per case.

| | | | Molecular | | |
|---|---|---|---|---|---|
| | | | weight | Immunostains | Immunostains |
| | Identified Proteins | Accession number | (kDa) | ISD066s1.mgf | ISD066s2.mgf |
| 1 | Ig gamma-2 chain | IGHG2_HUMAN | 36 | 100% | 99% |
| | C region | | | | |
| 2 | Ig heavy chain V- | HV305_HUMAN | 13 | 85% | 94% |
| | III region BRO | | | | |
| 3 | Fibrinogen beta | FIBB_HUMAN | 56 | | 100% |
| | chain precursor | | | | |
| 4 | Fibrinogen alpha | FIBA_HUMAN | 95 | | 100 |
| | chain precursor | | | | |
| 5 | Vimentin | VIME_HUMAN | 54 | 100% | 94% |
| 6 | Hemoglobin | HBB_HUMAN | 16 | 100% | 100% |
| | subunit beta | | | | |
| 7 | Glial fibrillary | GFAP_HUMAN | 50 | 100% | 100% |
| | acidic protein | | | | |
| 8 | Hemoglobin | HBA_HUMAN | 15 | 100% | 100% |
| | subunit alpha | | | | |
| 9 | Pumilio homolog 2 | PUM2_HUMAN | 114 | 85% | 98% |

TABLE 6
Scaffold Results for MS data case 3 with top 10 proteins identified by
probability based algorithm per case.

| | | | Molecular | | |
|---|---|---|---|---|---|
| | | | weight | Immunostains | Immunostains |
| | Identified Proteins | Accession number | (kDa) | SPA706307.mgf | SPA706308.mgf |
| 1 | Ig kappa chain C | KAC_HUMAN | 12 | 100% | 100% |
| | region | | | | |
| 2 | Ig gamma-1 chain | IGHG1_HUMAN | 36 | 100% | 100% |
| | C region | | | | |
| 3 | Apolipoprotein E | APOE_HUMAN | 36 | 100% | 100% |
| | precursor | | | | |
| 4 | Clusterin precursor | CLUS_HUMAN | 52 | 100% | 100% |
| 5 | Glial fibrillary | GFAP_HUMAN | 50 | 100% | 100% |
| | acidic protein | | | | |
| 6 | Collagen alpha-2(I) | CO1A2_HUMAN | 129 | 100% | 100% |
| | chain precursor | | | | |
| 7 | Collagen alpha-1(I) | CO1A1_HUMAN | 139 | 100% | 100% |
| | chain precursor | | | | |
| 8 | Serum albumin | ALBU-HUMAN | 69 | 100% | 100% |
| | precursor | | | | |
| 9 | Hemoglobin | HBB_HUMAN | 16 | 100% | 100% |
| | subunit beta | | | | |
| 10 | Vitronectin | VTNC_HUMAN | 54 | 100% | 100% |
| | precursor | | | | |

TABLE 7
Scaffold Results for MS data case 4 with top 10 proteins identified by
probability based algorithm per case.

| | | | Molecular | | |
|---|---|---|---|---|---|
| | | | weight | Immunostains | Immunostains |
| | Identified Proteins | Accession number | (kDa) | ISD062s1.mgf | ISD062s2.mgf |
| 1 | Ig gamma-1 chain | IGHG1_HUMAN | 36 | 100% | 100% |
| | C region | | | | |
| 2 | Ig kappa chain C | KAC_HUMAN | 12 | 100% | 100% |
| | region | | | | |
| 3 | Ig alpha-1 chain C | IGHA1_HUMAN | 38 | 100% | 92% |
| | region | | | | |
| 4 | Ig gamma-3 chain | IGHG3_HUMAN | 32 | | 92% |
| | C region | | | | |
| 5 | Serum albumin | ALBU-HUMAN | 69 | 100% | 100% |
| | precursor | | | | |
| 6 | Ryanodine receptor 3 | RYR3_HUMAN | 552 | 84% | 91% |
| 7 | NFX1-type zinc | ZNFX1_HUMAN | 220 | 94% | |
| | finger-containing | | | | |
| | protein | | | | |
| 8 | Latent-transforming | LTBP3_HUMAN | 139 | 94% | 92% |
| | growth factor | | | | |
| 9 | Mucin-16 | MUC16_HUMAN | 2353 | 94% | 92% |
| 10 | Neurogenic locus | NOTC4_HUMAN | 210 | 94% | 92% |
| | notch homolog | | | | |

TABLE 8
Scaffold Results for MS data case 5 with top 10 proteins identified by
probability based algorithm per case.

| | | | Molecular | | |
|---|---|---|---|---|---|
| | | | weight | Immunostains | Immunostains |
| | Identified Proteins | Accession number | (kDa) | SPA706309.mgf | SPA706310.mgf |
| 1 | Apolipoprotein E | APOE_HUMAN | 36 | 100% | 100% |
| | precursor | | | | |
| 2 | Serum amyloid P- | SAMP_HUMAN | 25 | 100% | 100% |
| | component | | | | |
| | precursor | | | | |
| 3 | Ig alpha-1 chain C | IGHA1-HUMAN | 38 | 100% | 100% |
| | region | | | | |
| 4 | Apolipoprotein A- | APOA4_HUMAN | 45 | 100% | 100% |
| | IV precursor | | | | |
| 5 | Ig lambda chain C | LAC_HUMAN | 11 | 100% | |
| | regions | | | | |
| 6 | Glial fibrillary | GFAP_HUMAN | 50 | 100% | 100% |
| | acidic protein | | | | |
| 7 | Vimentin | VIME_HUMAN | 54 | 100% | 100% |
| 8 | Vitronectin | VTNC_HUMAN | 54 | 100% | 100% |
| | precursor | | | | |
| 9 | Serum albumin | ALBU-HUMAN | 69 | 100% | 100% |
| | precursor | | | | |
| 10 | Clusterin precursor | CLUS_HUMAN | 52 | 100% | 100% |

TABLE 9
Scaffold Results for MS data case 6 with top 10 proteins identified by
probability based algorithm per case.

| | | | Molecular | | | Post- |
|---|---|---|---|---|---|---|
| | Identified | Accession | weight | Immunostains | Immunostains | immunostains |
| | Proteins | number | (kDa) | ISD157s1.mgf | ISD157s2.mgf | ISD157s2.mgf |
| 1 | Ig gamma-1 | IGHG1_HUMAN | 36 | 100% | 100% | |
| | chain C | | | | | |
| | region | | | | | |
| 2 | Ig kappa | KAC_HUMAN | 12 | 100% | | 90% |
| | chain C | | | | | |
| | region | | | | | |
| 3 | Serum | ALBU- | 69 | 100% | 100% | |
| | albumin | HUMAN | | | | |
| | precursor | | | | | |
| 4 | Vimentin | VIME_HUMAN | 54 | 100% | 100% | |
| 5 | Actin, | ACTB_HUMAN | 42 | 100% | 100% | |
| | cytoplasmic 1 | | | | | |
| 6 | Hemoglobin | HBA_HUMAN | 15 | 100% | 93% | |
| | subunit | | | | | |
| | alpha | | | | | |
| 7 | Ryanodine | RYR3_HUMAN | 552 | 84% | 91% | |
| | receptor 3 | | | | | |
| 8 | NF-X1-type | NFXL1_HUMAN | 101 | 98% | 87% | |
| | zinc finger | | | | | |
| | protein | | | | | |
| 9 | Vacuolar | VP13B_HUMAN | 449 | 91% | 93% | |
| | protein | | | | | |
| | sorting- | | | | | |
| | associated | | | | | |
| 10 | Metallothionein-4 | MT4_HUMAN | 6 | 74% | 93% | |

TABLE 10
Scaffold Results for MS data case 8 with top 10 proteins identified by
probability based algorithm per case.

| | | Accession | Molecular | Immunostains | Immunostains |
|---|---|---|---|---|---|
| | Identified Proteins | number | weight (kDa) | ISD160s1.mgf | ISD160s2.mgf |
| 1 | Apolipoprotein E | APOE_HUMAN | 36 | 100% | 100% |
| | precursor | | | | |
| 2 | Apolipoprotein A-IV | APOA4_HUMAN | 45 | 100% | 100% |
| | precursor | | | | |
| 3 | Ig lambda chain V-IV | LV4A_HUMAN | 11 | 94% | 94% |
| | region | | | | |
| 4 | Serum amyloid P- | SAMP_HUMAN | 25 | 100% | 100% |
| | component precursor | | | | |
| 5 | Apolipoprotein A-I | APOA1_HUMAN | 31 | 94% | 100% |
| | precursor | | | | |
| 6 | Hemoglobin subunit | HBB_HUMAN | 16 | 100% | 100% |
| | beta | | | | |
| 7 | Vitronectin precursor | VTNC_HUMAN | 54 | 100% | 100% |
| 8 | Glial fibrillary acidic | GFAP_HUMAN | 50 | 100% | 100% |
| | protein | | | | |
| 9 | Clusterin precursor | CLUS_HUMAN | 52 | 100% | 100% |
| 10 | Low density | LRP1B_HUMAN | 515 | 99% | 98% |
| | lipoprotein | | | | |

TABLE 11
Scaffold Results for MS data case 9 with top 10 proteins identified by
probability based algorithm per case.

| | | Accession | Molecular | Immunostains | Immunostains |
|---|---|---|---|---|---|
| | Identified Proteins | number | weight (kDa) | ISD160s1.mgf | ISD160s2.mgf |
| 1 | Ig mu chain C-region | MUC_HUMAN | 50 | 100% | 100% |
| 2 | Ig kappa chain V-III | KV312_HUMAN | 14 | 100% | 100% |
| | region HAH | | | | |
| 3 | Apolipoprotein A-I | APOA1_HUMAN | 31 | 90% | 100% |
| | precursor | | | | |
| 4 | Ig kappa chain C | KAC_HUMAN | 12 | 90% | 100% |
| | region | | | | |
| 5 | Immunoglobulin J | IgJ_HUMAN | 16 | 100% | 100% |
| | chain | | | | |
| 6 | Ig kappa chain V-III | KV305_HUMAN | 12 | 90% | 89% |
| | region WOL | | | | |
| 7 | Ig heavy chain V-I | HV103_HUMAN | 13 | | 89% |
| | region V35 | | | | |
| 8 | Serum albumin | ALBU- | 69 | 100% | 100% |
| | precursor | HUMAN | | | |
| 9 | Hemoglobin subunit | HBB_HUMAN | 16 | 100% | 93% |
| | beta | | | | |
| 10 | Hemoglobin subunit | HBA_HUMAN | 15 | 100% | 93% |
| | alpha | | | | |

TABLE 12
Scaffold Results for MS data case 10 with top 10 proteins identified by
probability based algorithm per case.

| | | | Molecular | Immunostains | Immunostains | Immunostains |
|---|---|---|---|---|---|---|
| | Identified | Accession | weight | ISD159_B1_S1. | ISD159_B1_S3. | ISD159_B1_S3. |
| | Proteins | number | (kDa) | mgf | mgf | mgf |
| 1 | Apolipoprotein | APOA4_HUMAN | 45 | | 100% | 100% |
| | A- | | | | | |
| | IV | | | | | |
| | precursor | | | | | |
| 2 | Ig lambda | LAC_HUMAN | 11 | 86% | 100% | 100% |
| | chain C | | | | | |
| | regions | | | | | |
| 3 | Apolipoprotein | APOA1_HUMAN | 31 | | 100% | 94% |
| | A-I | | | | | |
| | precursor | | | | | |
| 4 | Serum | SAMP_HUMAN | 25 | | 100% | 94% |
| | amyloid | | | | | |
| | P- | | | | | |
| | component | | | | | |
| | precursor | | | | | |
| 5 | Ig alpha-1 | IGHA1- | 38 | | 100% | 94% |
| | chain C | HUMAN | | | | |
| | region | | | | | |
| 6 | Ig | IGHG1_HUMAN | 36 | | 94% | 94% |
| | gamma-1 | | | | | |
| | chain C | | | | | |
| | region | | | | | |
| 7 | Vimentin | VIME_HUMAN | 54 | 100% | 100% | |
| 8 | Glial | GFAP_HUMAN | 50 | 100% | 100% | |
| | fibrillary | | | | | |
| | acidic | | | | | |
| | protein | | | | | |
| 9 | Apolipoprotein E | APOE_HUMAN | 36 | 100% | 100% | |
| | precursor | | | | | |
| 10 | Actin, | ACTB_HUMAN | 42 | | 100% | 100% |
| | cytoplasmic 1 | | | | | |

TABLE 13
Scaffold Results for MS data case 11 with top 10 proteins identified by
probability based algorithm per case.

| | | Accession | Molecular weight | Immunostains |
|---|---|---|---|---|
| | Identified Proteins | number | (kDa) | ISD161 combined |
| 1 | Ig alpha-1 chain C | IGHA1-HUMAN | 38 | 100% |
| | region | | | |
| 2 | Ig kappa chain V-III | KV3B_HUMAN | 12 | 100% |
| | region | | | |
| 3 | Ig kappa chain C | KAC_HUMAN | 12 | 100% |
| | region | | | |
| 4 | Apolipoprotein A-I | APOA1_HUMAN | 31 | 100% |
| | precursor | | | |
| 5 | Immunoglobulin J | IgJ_HUMAN | 16 | 100% |
| | chain | | | |
| 6 | Fibrinogen alpha chain | FIBA_HUMAN | 95 | 100% |
| | precursor | | | |
| 7 | Ig gamma-1 chain C | IGHG1_HUMAN | 36 | 100% |
| | region | | | |
| 8 | Amyloid beta A4 | APBB2_HUMAN | 83 | 92% |
| | precursor | | | |
| 9 | Apolipoprotein A-II | APOA2_HUMAN | 11 | 92% |
| | precursor | | | |
| 10 | Fibrinogen beta chain | FIBB_HUMAN | 56 | 92% |
| | precursor | | | |

TABLE 14
Scaffold Results for MS data case 12 with top 10 proteins identified by
probability based algorithm per case.

| | | | Molecular | | | |
|---|---|---|---|---|---|---|
| | Identified | Accession | weight | Immunostains | Immunostains | Immunostains |
| | Proteins | number | (kDa) | ISD155s1.mgf | ISD155s2.mgf | ISD155s3.mgf |
| 1 | Apolipoprotein | APOA4_HUMAN | 45 | 100% | | 100% |
| | A-IV | | | | | |
| | precursor | | | | | |
| 2 | Serum | SAMP_HUMAN | 25 | | 92% | 100% |
| | amyloid P- | | | | | |
| | component | | | | | |
| | precursor | | | | | |
| 3 | Apolipoprotein | APOA1_HUMAN | 31 | 100% | | 100% |
| | A-I | | | | | |
| | precursor | | | | | |
| 4 | Apolipoprotein E | APOE_HUMAN | 36 | 93% | | 98% |
| | precursor | | | | | |
| 5 | Ig lambda | LV603_HUMAN | 12 | 93% | | 91% |
| | chain V-VI | | | | | |
| | region SUT | | | | | |
| 6 | Ig lambda | LAC_HUMAN | 11 | 93% | | |
| | chain C | | | | | |
| | regions | | | | | |
| 7 | Serum | SAA4_HUMAN | 15 | | | 91% |
| | amyloid A-4 | | | | | |
| | protein | | | | | |
| | precursor | | | | | |
| 8 | Hemoglobin | HBB_HUMAN | 16 | 100% | 100% | 100% |
| | subunit beta | | | | | |
| 9 | Serum | ALBU_HUMAN | 69 | 100% | 100% | 100% |
| | albumin | | | | | |
| | precursor | | | | | |
| 10 | Collagen | CO1A2_HUMAN | 129 | 100% | 100% | 100% |
| | alpha 2(I) | | | | | |
| | chain | | | | | |
| | precursor | | | | | |

TABLE 15
Scaffold Results for MS data case 13 with top 10 proteins identified by
probability based algorithm per case.

| | | | Molecular | | |
|---|---|---|---|---|---|
| | Identified | Accession | weight | Immunostains | Immunostains |
| | Proteins | number | (kDa) | ISD170s1 | ISD170s2 |
| 1 | Apolipoprotein E | APOE_HUMAN | 36 | 100% | 94% |
| | precursor | | | | |
| 2 | Ig mu chain | MUC_HUMAN | 50 | 100% | |
| | C-region | | | | |
| 3 | Ig gamma-1 | IGHG1_HUMAN | 36 | 100% | |
| | chain C | | | | |
| | region | | | | |
| 4 | Complement | CO9_HUMAN | 63 | 93% | 94% |
| | component | | | | |
| | C9 precursor | | | | |
| 5 | Ig kappa | KAC_HUMAN | 12 | 100% | |
| | chain C | | | | |
| | region | | | | |
| 6 | Ig lambda | LAC_HUMAN | 11 | 100% | |
| | chain C | | | | |
| | regions | | | | |
| 7 | Apolipoprotein | APOA1_HUMAN | 31 | 99% | |
| | A-I | | | | |
| | precursor | | | | |
| 8 | Ig heavy | HV308_HUMAN | 13 | | 94% |
| | chain V-III | | | | |
| | region GA | | | | |
| 9 | Ig gamma-2 | IGHG2_HUMAN | 36 | 93% | |
| | chain C | | | | |
| | region | | | | |
| 10 | Ig gamma-3 | IGHG3_HUMAN | 32 | 93% | |
| | chain C | | | | |
| | region | | | | |

### Extracellular Proteinaceous Deposit Not Otherwise Specified

Interestingly, the non-amyloid extracellular depositions contained kappa light chains, but not lambda light chains in most cases (n=4). Two cases contained kappa as well as lambda light chains. Additional proteins identified included IgG (n=5), IgM (n=2), IgA (n=2), IgJ (n=2), Ig heavy chain (n=2), Apolipoprotein A-I (ApoA-I) (n=3), and ApoE (n=2). ApoA-IV, ApoA-II, and C9 were identified in single cases each. Immunohistochemistry with kappa and lambda light chain confirmed kappa light chain restriction in 4 cases, either in the deposits (n=3) and/or the inflammatory infiltrate (n=2). A polytypic pattern of light chain expression by immunohistochemistry was present in one (of two) cases demonstrating both kappa and lambda light chain peptides by LC-MS/MS. In addition, one (of two) cases with IgM showed convincing immunostaining (FIG. 16).

### Amyloidomas

Lambda light chains, SAP, ApoE and ApoA-IV were identified in all cases (n=5). In addition, apoA-I was identified in three cases, and IgG and IgA in single cases each. Immunohistochemistry demonstrated lambda (but not kappa) light chains in the aggregates in all cases, and SAP in 2 (of 3) cases tested.

### Intracellular Crystals

The example consistent with crystal storing histiocytosis (case 7) demonstrated kappa light chain as well as a minor GFAP component on LC-MS/MS. The GFAP was likely secondary to the presence of underlying brain tissue/reactive astrocytes in the microdissected area. Immunostains for kappa and lambda light chains were non-contributory secondary to ample background staining However, in situ hybridization labeled monotypic kappa plasma cells (FIG. 18). Conversely, in case 2, lambda light chain, IgG, and Ig heavy chain were identified by LC-MS/MS. The crystals demonstrated reactivity with IgG and lambda light chains, but not kappa, antibodies by immunohistochemistry.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for evaluating amyloidosis in a mammal, wherein said method comprises determining the identity of an amyloid polypeptide present in a fat aspirate sample from said mammal using mass spectrometry, wherein said fat aspirate sample was not processed using liquid chromatography before performing said mass spectrometry.

2. The method of claim 1, wherein said mammal is a human.

3. The method of claim 1, wherein said liquid chromatography is high-performance liquid chromatography.

4. The method of claim 1, wherein said amyloid polypeptide is a transthyretin polypeptide, a serum amyloid A polypeptide, a lambda light chain polypeptide, a kappa light chain polypeptide, a serum amyloid P polypeptide, a LECT2 polypeptide, a heavy chain polypeptide, a fibrinogen alpha polypeptide, a gelsolin polypeptide, a beta2 microglobulin polypeptide, an apoplipoprotein AI or AII polypeptide, or a lysozyme polypeptide.

5. The method of claim 1, wherein said fat aspirate sample was treated with a protease before said mass spectrometry.

6. The method of claim 5, wherein said protease is trypsin.

7. A method for evaluating amyloidosis in a mammal, wherein said method comprises
determining whether or not a tissue sample Congo Red positive for amyloid from said mammal comprises a transthyretin polypeptide using mass spectrometry,
determining whether or not a tissue sample Congo Red positive for amyloid from said mammal comprises a serum amyloid A polypeptide using mass spectrometry,
determining whether or not a tissue sample Congo Red positive for amyloid from said mammal comprises a lambda light chain polypeptide using mass spectrometry, and
determining whether or not a tissue sample Congo Red positive for amyloid from said mammal comprises a kappa light chain polypeptide using mass spectrometry.

8. The method of claim 7, wherein said method comprises determining whether or not a tissue sample Congo Red positive for amyloid from said mammal comprises a LECT2 polypeptide using mass spectrometry.

9. The method of claim 7, wherein said mammal is a human.

10. The method of claim 7, wherein said tissue sample was treated with a protease before said mass spectrometry.

11. The method of claim 10, wherein said protease is trypsin.
